(19) United States
(12) Patent Application Publication
(10) Pub. No.: US 2009/0318514 A1
(43) Pub. Date: Dec. 24, 2009

(54) Novel Pharmaceutical Compositions

(76) Inventors: Ana Maria Garcia Collazo, Madrid (ES); Eva Kristina Koch, Stockholm (SE); Anton Joakim Löfstedt, Uppsala (SE); Aiping Cheng, Huddinge (SE); Sandra Gordon, Mariefred (SE); Tomas Fredrik Hansson, Tumba (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/226,841

(22) PCT Filed: May 3, 2007
(86) PCT No.: PCT/EP2007/003904

§ 371 Date: Mar. 16, 2009

(30) Foreign Application Priority Data
May 3, 2006 (GB) 06 08724.1

(51) Int. Cl. A61K 31/422; C07D 235/12; C07D 235/16; C07D 263/30; A61K 31/4184; A61P 5/14

(52) U.S. Cl. 514/374; 548/310.1; 548/309.1; 548/235; 514/394

(57) ABSTRACT

The invention provides the use of compounds of formula (I) or pharmaceutically acceptable esters, amides, solvates or salts thereof, including salts of such esters or amides, and solvates of such esters, amides or salts, for the manufacture of a medicament for the treatment or prophylaxis of a condition that may be treated with a thyroid receptor agonist or partial agonist wherein R¹, R², R³, R⁴, Y, W and R⁵ are as defined in the specification. The invention also provides compounds of formula (Ia) or pharmaceutically acceptable esters, amides, solvates or salts thereof, including salts of such esters or amides, and solvates of such esters, amides or salts, formula (Ia) wherein R¹, R², R³, R⁴, Y, W and R⁵ are as defined in the specification.

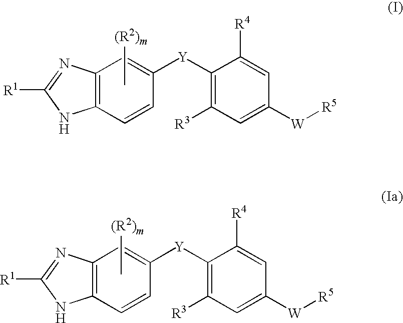

### FIELD OF THE INVENTION

The present invention relates to compounds which are agonists or partial agonists of the thyroid receptor and the use of such compounds for therapeutic purposes.

### BACKGROUND OF THE INVENTION

While the extensive role of thyroid hormones in regulating metabolism in humans is well recognized, the discovery and development of new specific drugs for improving the treatment of hyperthyroidism and hypothyroidism has been slow. This has also limited the development of thyroid agonists and antagonists for treatment of other important clinical indications, such as hypercholesterolemia, dyslipidemia, obesity, diabetes, atherosclerosis, cardiac diseases and various endocrine disorders.

Thyroid hormones affect the metabolism of virtually every cell of the body. At normal levels, these hormones maintain body weight, metabolic rate, body temperature and mood, and influence blood levels of serum lipoproteins. Thus, in hypothyroidism there is weight gain, high levels of LDL cholesterol, and depression. In hyperthyroidism, these hormones lead to weight loss, hypermetabolism, lowering of serum LDL cholesterol levels, cardiac arrhythmias, heart failure, muscle weakness, bone loss in postmenopausal women, and anxiety.

Thyroid hormones are currently used primarily as replacement therapy for patients with hypothyroidism. Therapy with thyroxine (3,5,3′,5′-tetraiodo-L-thyronine, or T₄) and triiodothyronine (3,5,3′-triiodo-L-thyronine, or T₃) returns metabolic functions to normal and can easily be monitored with routine serum measurements of levels of thyroid-stimulating hormone (TSH), T₄ or T₃. However, replacement therapy, particularly in older individuals, may be restricted by certain detrimental effects from thyroid hormones.

In addition, some effects of thyroid hormones may be therapeutically useful in non-thyroid disorders if adverse effects can be minimized or eliminated. These potentially useful influences include for example, lowering of serum LDL levels, weight reduction, amelioration of depression and stimulation of bone formation. Prior attempts to utilize thyroid hormones pharmacologically to treat these disorders have been limited by manifestations of hyperthyroidism, and in particular by cardiovascular toxicity.

Furthermore, useful thyroid agonist drugs should minimize the potential for undesired consequences due to locally induced hypothyroidism, i.e. sub-normal levels of thyroid hormone activity in certain tissues or organs. This can arise because increased circulating thyroid hormone agonist concentrations may cause the pituitary to suppress the secretion of thyroid stimulating hormone (TSH), thereby reducing thyroid hormone synthesis by the thyroid gland (negative feedback control). Since endogenous thyroid hormone levels are reduced, localized hypothyroidism can result wherever the administered thyroid agonist drug fails to compensate for the reduction in endogenous hormone levels in specific tissues.

Development of specific and selective thyroid hormone receptor ligands, particularly agonists of the thyroid hormone receptor, is expected to lead to specific therapies for these common disorders, while avoiding the cardiovascular and other toxicity of native thyroid hormones. Tissue-selective thyroid hormone agonists may be obtained by selective tissue uptake or extrusion, topical or local delivery, targeting to cells through other ligands attached to the agonist and targeting receptor subtypes. Tissue selectivity can also be achieved by selective regulation of thyroid hormone responsive genes in a tissue specific manner.

Certain compounds which are thyroid receptor ligands are described in WO 02/062780. The compounds are described as antagonists or partial antagonists of the thyroid hormone receptor, which may be used in the treatment of cardiac and metabolic disorders, such as cardiac arrhythmias, thyrotoxicosis, subclinical hyperthyroidism and liver diseases.

The compounds that are thyroid hormone receptor ligands, particularly selective agonists of the thyroid hormone receptor, are expected to demonstrate a utility for the treatment or prevention of diseases or disorders associated with thyroid hormone activity, for example: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes; (7) depression; (8) osteoporosis (especially in combination with a bone resorption inhibitor); (9) goiter; (10) thyroid cancer; and (11) glaucoma.

### SUMMARY OF THE INVENTION

In a first aspect, the present invention provides the use of a compound of formula (I) or a pharmaceutically acceptable ester, amide, solvate or salt thereof, including a salt of such an ester or amide, and a solvate of such an ester, amide or salt, for the manufacture of a medicament for the treatment or prophylaxis of a condition that may be treated with a thyroid receptor agonist or partial agonist

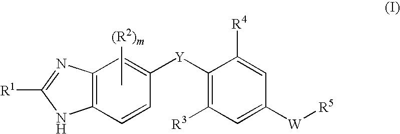

wherein:

R¹ is selected from halogen, N(R^{b})₂, ≦CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, —CH₂)ₙ—CO—N(R^{a})₂, —CO₂H, C₁₋₈ alkyl, C₂₋₄ alkenyl, C₂₋₄ alkynyl, fluoromethyl, difluoromethyl, trifluoromethyl, C₃₋₆ cycloalkyl, C₃₋₆ cycloalkyl-C₁₋₃ alkyl, phenyl, benzyl and C₃₋₇ heterocyclyl, said alkyl, alkenyl or alkynyl groups or portions of groups optionally being substituted with 1, 2, 3, 4 or 5 groups each independently selected from halogen, hydroxy, C₁₋₄ alkylthio, N(R^{b})₂, phenyl, methoxy, halomethoxy, dihalomethoxy and trihalomethoxy; said cycloalkyl, phenyl, benzyl or heterocyclyl groups or portions of groups optionally being substituted with 1, 2 or 3 groups independently selected from halogen, hydroxy, C₁₋₄ alkyl, C₂₋₄ alkenyl, C₂₋₄ alkynyl, N(R^{b})₂, methoxy, haloC₁₋₄alkyl, dihaloC₁₋₄alkyl, trihaloC₁₋₄alkyl, halomethoxy, dihalomethoxy, and trihalomethoxy;

R^{a} is independently selected from C₁₋₄ alkyl, C₂₋₄ alkenyl, C₂₋₄ alkynyl, benzyl, heterocyclyl and phenyl, said phenyl group or portion of group optionally being substituted with 1, 2 or 3 groups independently selected from C₁₋₄ alkyl, halogen, hydroxy, methoxy, halomethoxy, dihalomethoxy, and trihalomethoxy; said alkyl, alkenyl, or alkynyl groups or portions of groups optionally being substituted with 1, 2 or 3 groups independently selected from halogen, hydroxy, methoxy, halomethoxy, dihalomethoxy, and trihalomethoxy;

n is 0, 1, 2 or 3;

Each R² is independently selected from halogen, hydroxy, cyano, C₁₋₄ alkoxy, C₁₋₄ alkyl and N(R^{b})₂, said alkyl or alkoxy groups or portions of groups optionally being substituted with 1, 2 or 3 groups selected from halogen, hydroxyl or C₁₋₄ alkoxy;

R^{b} is independently selected from hydrogen, C₁₋₄ alkyl C₂₋₄ alkenyl, and C₂₋₄ alkynyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy;

m is 0, 1 or 2;

Y is selected from oxygen, methylene, sulphur, N(R^{b})₂, —S(O)— and —S(O)₂—;

R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;

W is selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene—O—C₁₋₃ alkylene, C(O)NH—C₁₋₃ alkylene, NHC(O)—C₀₋₃ alkylene and C₁₋₃ alkyleneC(O)NH—C₁₋₃ alkylene, said alkylene, alkenylene or alkynylene groups being straight chain, and said alkylene, alkenylene or alkynylene groups or portions of groups optionally being substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;

R^{c} is selected from hydrogen, C₁₋₄ alkyl, C₂₋₄ alkenyl, C₂₋₄ alkynyl, fluoromethyl, difluoromethyl and trifluoromethyl;

R⁵ is selected from —CO₂R^{d}, —CONHR^{d}, —PO(OR^{d})₂, —PO(OR^{d})NH₂, —SO₂OR^{d}, —COCO₂R^{d}, —CONR^{d}OR^{d}, —SO₂NHR^{d}, —NHSO₂R^{d}, —CONHSO₂R^{d}, and —SO₂NHCOR^{d};

or WR⁵ together form the group NHCOR^{d}

Each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen and C₁₋₄ alkyl;

with the proviso that when Y is oxygen, W is methylene, m is 0, R³ and R⁴ are both chlorine, and R⁵ is CO₂H, R¹ is not isopropyl;

and with the further proviso that when Y is oxygen, W is methylene, m is 0, R³ and R⁴ are both bromine, and R⁵ is CO₂H, R¹ is not methyl.

In a second aspect, the present invention provides a compound of formula (Ia) or a pharmaceutically acceptable ester, amide, solvate or salt thereof, including a salt of such an ester or amide, and a solvate of such an ester, amide or salt,

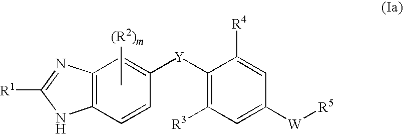

wherein:

R¹ is selected from halogen, N(R^{b})₂, —(CH₂)ₙ—NH—SO₂—R^{a}, —(CH₂)ₙ—SO₂—NH—R^{a}, —CH₂)ₙ—NH—CO—R^{a}, —CH₂)ₙ—CO—NH—R^{a}, —CH₂)ₙ—CO—N(R^{a})₂, —CO₂H, C₁₋₈ alkyl, C₂₋₄ alkenyl, C₂₋₄ alkynyl, fluoromethyl, difluoromethyl, trifluoromethyl, C₃₋₆ cycloalkyl, C₃₋₆ cycloalkyl-C₁₋₃ alkyl, phenyl, benzyl and C₃₋₇ heterocyclyl, said alkyl, alkenyl or alkynyl groups or portions of groups optionally being substituted with 1, 2, 3, 4 or 5 groups each independently selected from halogen, hydroxy, C₁₋₄ alkylthio, N(R^{b})₂, phenyl, methoxy, halomethoxy, dihalomethoxy and trihalomethoxy; said cycloalkyl, phenyl, benzyl or heterocyclyl groups or portions of groups optionally being substituted with 1, 2 or 3 groups independently selected from halogen, hydroxy, C₁₋₄ alkyl, C₂₋₄ alkenyl, C₂₋₄ alkynyl, N(R^{b})₂, methoxy, haloC₁₋₄alkyl, dihaloC₁₋₄alkyl, trihaloC₁₋₄alkyl, halomethoxy, dihalomethoxy, and trihalomethoxy;

R^{a} is independently selected from C₁₋₄ alkyl, C₂₋₄ alkenyl, C₂₋₄ alkynyl, benzyl, heterocyclyl and phenyl, said phenyl group or portion of group optionally being substituted with 1, 2 or 3 groups independently selected from C₁₋₄ alkyl, halogen, hydroxy, methoxy, halomethoxy, dihalomethoxy, and trihalomethoxy; said alkyl, alkenyl, or alkynyl groups or portions of groups optionally being substituted with 1, 2 or 3 groups independently selected from halogen, hydroxy, methoxy, halomethoxy, dihalomethoxy, and trihalomethoxy;

n is 0, 1, 2 or 3;

Each R² is independently selected from halogen, hydroxy, cyano, C₁₋₄ alkoxy, C₁₋₄ alkyl and N(R^{b})₂, said alkyl or alkoxy groups or portions of groups optionally being substituted with 1, 2 or 3 groups selected from halogen, hydroxyl or C₁₋₄ alkoxy;

R^{b} is independently selected from hydrogen, C₁₋₄ alkyl, C₂₋₄ alkenyl, and C₂₋₄ alkynyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy;

m is 0, 1 or 2;

Y is selected from oxygen, methylene, N(R^{b})₂, sulphur, —S(O)— and —S(O)₂—;

R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;

W is selected from
C₁ alkylene substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
Straight chain C₂₋₃ alkylene substituted with 1 or 2 groups selected from mercapto, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
C₂₋₃ alkenylene, C₂₋₃ alkynylene, N(R^{c})-C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NHC(O)—C₀₋₃ alkylene and C₁₋₃ alkyleneC(O)NH—C₁₋₃ alkylene, said alkylene, alkenylene or alkynylene groups being straight chain, and said alkylene, alkenylene or alkynylene groups or portions of groups optionally being substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;

R^{c} is selected from hydrogen, C₁₋₄ alkyl, C₂₋₄ alkenyl, C₂₋₄ alkynyl, fluoromethyl, difluoromethyl and trifluoromethyl;

R⁵ is selected from —CO₂R^{d}, —CONHR^{d}, —PO(OR^{d})₂, —PO(OR^{d})NH₂, —SO₂OR^{d}, —COCO₂R^{d}, —CONR^{d}OR^{d}, —SO₂NR^{d}, —NHSO₂R^{d}, —CONHSO₂R^{d}, and —SO₂NHCOR^{d};

or WR⁵ together form the group NHCOR^{d}

Each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen and C₁₋₄ alkyl.

In a third aspect, the present invention provides a compound of formula (Ib) or a pharmaceutically acceptable ester, amide, solvate or salt thereof, including a salt of such an ester or amide, and a solvate of such an ester, amide or salt,

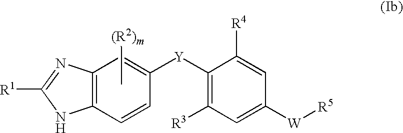

wherein:

R¹ is selected from
C₁₋₄ alkyl substituted with one group independently selected from halogen, hydroxy, C₁₋₄ alkylthio, N(R^{b})₂, methoxy, halomethoxy, dihalomethoxy and trihalomethoxy, and optionally substituted with 1, 2, 3 or 4 additional groups each independently selected from halogen, hydroxy, C₁₋₄ alkylthio, N(R^{b})₂, phenyl, methoxy, halomethoxy, dihalomethoxy and trihalomethoxy;
phenyl or C₅₋₇ heteroaryl, said phenyl or C₅₋₇ heteroaryl group being substituted with one group independently selected from chlorine, bromine, iodine, C₁₋₄ alkyl, C₂₋₄ alkenyl, C₂₋₄ alkynyl, haloC₁₋₄alkyl, dihaloC₁₋₄alkyl, trihaloC₁₋₄alkyl, halomethoxy, dihalomethoxy, and trihalomethoxy, and optionally substituted with 1 or 2 additional groups each independently selected from halogen, hydroxy, C₁₋₄ alkyl, C₂₋₄ alkenyl, C₂₋₄ alkynyl, N(R^{b})₂, methoxy, haloC₁₋₄alkyl, dihaloC₁₋₄alkyl, trihaloC₁₋₄alkyl, halomethoxy, dihalomethoxy, and trihalomethoxy;
halogen, N(R^{b})₂, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, C₅₋₈ alkyl, C₂₋₄ alkenyl, C₂₋₄ alkynyl, fluoromethyl, difluoromethyl, trifluoromethyl, C₃₋₆ cycloalkyl, C₃₋₆ cycloalkyl-C₁₋₃ alkyl, benzyl and C₃₋₄ heterocyclyl, C₅₋₇ heterocycloalkyl, said alkyl, alkenyl or alkynyl groups or portions of groups optionally being substituted with 1, 2 or 3 groups each independently selected from halogen, hydroxy, C₁₋₄ alkylthio, N(R^{b})₂, phenyl, methoxy, halomethoxy, dihalomethoxy and trihalomethoxy; said cycloalkyl, benzyl, heterocyclyl or heterocycloalkyl groups or portions of groups optionally being substituted with 1, 2 or 3 groups independently selected from halogen, hydroxy, C₁₋₄ alkyl, C₂₋₄ alkenyl, C₂₋₄ alkynyl, N(R^{b})₂, methoxy, halomethoxy, dihalomethoxy, and trihalomethoxy;

R^{a} is independently selected from C₁₋₄ alkyl, C₂₋₄ alkenyl, C₂₋₄ alkynyl, benzyl, heterocyclyl and phenyl, said phenyl group or portion of group optionally being substituted with 1, 2 or 3 groups independently selected from C₁₋₄ alkyl, halogen, hydroxy, methoxy, halomethoxy, dihalomethoxy, and trihalomethoxy; said alkyl, alkenyl, or alkynyl groups or portions of groups optionally being substituted with 1, 2 or 3 groups independently selected from halogen, hydroxy, methoxy, halomethoxy, dihalomethoxy, and trihalomethoxy;

n is 0, 1, 2 or 3;

Each R² is independently selected from halogen, hydroxy, cyano, C₁₋₄ alkoxy, C₁₋₄ alkyl and N(R^{b})₂, said alkyl or alkoxy groups or portions of groups optionally being substituted with 1, 2 or 3 groups selected from halogen, hydroxyl or C₁₋₄ alkoxy;

R^{b} is independently selected from hydrogen, C₁₋₄ alkyl, C₂₋₄ alkenyl, and C₂₋₄ alkynyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy;

m is 0, 1 or 2;

Y is selected from oxygen, methylene, N(R^{b})₂, sulphur, —S(O)— and —S(O)₂—;

R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;

W is selected from C₁₋₃ alkylene, and C₂₋₃ alkylene substituted with 1 or 2 groups selected from hydroxy and amino;

R⁵ is selected from —CO₂R^{d}, —CONHR^{d}, —PO(OR^{d})₂, —PO(OR^{d})NH₂, —SO₂OR^{d}, —COCO₂R^{d}, —CONR^{d}OR^{d}, —SO₂NHR^{d}, —NHSO₂R^{d}, —CONHSO₂R^{d}, and —SO₂NHCOR^{d};

or WR⁵ together form the group NHCOR^{d}

Each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen and C₁₋₄ alkyl.

In a fourth aspect, the present invention provides compound of formula (Ic) or a pharmaceutically acceptable ester, amide, solvate or salt thereof, including a salt of such an ester or amide, and a solvate of such an ester, amide or salt,

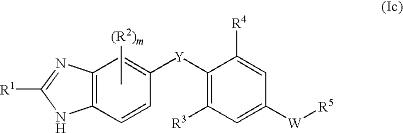

wherein:

R¹ is selected from C₁₋₄ alkyl, phenyl, and C₅₋₇ heteroaryl, said alkyl groups optionally being substituted with 1, 2 or 3 phenyl groups; said phenyl or heteroaryl groups optionally being substituted with 1, 2 or 3 groups independently selected from fluorine, hydroxy, methoxy and N(R^{b})₂;

Each R² is independently selected from halogen, cyano, hydroxy, C₁₋₄ alkoxy, C₁₋₄ alkyl and N(R^{b})₂, said alkyl or alkoxy groups or portions of groups optionally being substituted with 1, 2 or 3 groups selected from halogen, hydroxyl or C₁₋₄ alkoxy;

R^{b} is independently selected from hydrogen, C₁₋₄ alkyl, C₂₋₄ alkenyl, and C₂₋₄ alkynyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy;

m is 0, 1 or 2;

Y is selected from oxygen, methylene, N(R^{b})₂, sulphur, —S(O)— and —S(O)₂—;

R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;

W is selected from C₁₋₃ alkylene, and C₂₋₃ alkylene substituted with 1 or 2 groups selected from hydroxy and amino;

R⁵ is selected from —CO₂R^{d}, —CONHR^{d}, —PO(OR^{d})₂, —PO(OR^{d})NH₂, —SO₂OR^{d}, —COCO₂R^{d}, —CONR^{d}OR^{d}, —SO₂NHR^{d}, —NHSO₂R^{d}, —CONHSO₂R^{d}, and -SO₂NHCOR^{d};

Each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen and C₁₋₄ alkyl;

with the proviso that when Y is oxygen, W is methylene, m is 0, R³ and R⁴ are both chlorine, and R⁵ is CO₂H, R¹ is not isopropyl;

and with the further proviso that when Y is oxygen, W is methylene, m is 0, R³ and R⁴ are both bromine, and R⁵ is CO₂H, R¹ is not methyl.

Compounds of the invention have surprisingly been found to be ligands of the thyroid receptor, in particular agonists or partial agonists of the thyroid receptor. The compounds accordingly have use in the treatment or prophylaxis of conditions associated with thyroid receptor activity, in particular the compounds have use in the treatment or prophylaxis of conditions that may be treated with a thyroid receptor agonist or partial agonist.

### DETAILED DESCRIPTION OF THE INVENTION

The compounds of formula (I) may contain chiral (asymmetric) centres or the molecule as a whole may be chiral. The individual stereoisomers (enantiomers and diastereoisomers) and mixtures of these are within the scope of the present invention.

For compounds of formula (I) or (Ia), R¹ is preferably selected from —CH₂)ₙ—NH—SO₂—R^{a}, —CH₂)ₙ—SO₂—NH—R^{a}, —(CH₂)ₙ—NH—CO—R^{a}, —(CH₂)ₙ—CO—NH—R^{a}, —(CH₂)ₙ—CO—N(R^{a})₂, C₁₋₈ alkyl, C₂₋₄ alkenyl, C₃₋₆ cycloalkyl, C₃₋₆ cycloalkyl-C₁₋₃ alkyl, phenyl, benzyl and C₃₋₇ heterocyclyl.

For compounds of formula (I) or (Ia), when m is 0, and simultaneously both R³ and R⁴ are bromine, R¹ is preferably selected from, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, C₂₋₈ alkyl, C₂₋₄ alkenyl, C₃₋₆ cycloalkyl, C₃₋₆ cycloalkyl-C₁₋₃ alkyl, phenyl, benzyl and C₃₋₇ heterocyclyl.

For compounds of formula (I) or (Ia), when m is 0, and simultaneously both R³ and R⁴ are chlorine, R¹ is preferably selected —(CH₂)ₙ—NH—SO₂—R^{a}, —(CH₂)ₙ—SO₂—NH—R^{a}, —CH₂)ₙ—NH—CO—R^{a}, —CH₂)ₙ—CO—NH—R^{a}, methyl, ethyl, n-propyl, C₄₋₈ alkyl, C₂₋₄ alkenyl, C₃₋₆ cycloalkyl, C₃₋₆ cycloalkyl—C₁₋₃ alkyl, phenyl, benzyl and C₃₋₇ heterocyclyl.

For compounds of formula (I) or (Ia), R¹ is more preferably selected from —CH₂)ₙ—NH—SO₂—R^{a}, —(CH₂)ₙ—SO₂—NH—R^{a}, —(CH₂)ₙ—NH—CO—R^{a}, —CH₂)ₙ—CO—NH—R^{a}, C₂₋₅ alkyl, phenyl, benzyl, C₃₋₅ cycloalkyl-C₁₋₃ alkyl and C₃₋₅ heterocyclyl. Preferred substituents for said alkyl include groups independently selected from halogen, hydroxyl, C₁₋₄ alkylthio, N(R^{b})₂ and methoxy. Preferred substituents for said cycloalkyl, phenyl, benzyl or heterocyclyl include groups independently selected from halogen, hydroxy, C₁₋₄ alkyl, trifluoromethyl, N(R^{b})₂, methoxy, haloC₁₋₄alkyl, dihaloC₁₋₄alkyl, trihaloC₁₋₄alkyl, halomethoxy, dihalomethoxy, and trihalomethoxy.

For compounds of formula (Ib), in one embodiment, R¹ is preferably selected from —CH₂)ₙ—NH—SO₂—R^{a}, —(CH₂)ₙ—SO₂—NH—R^{a}, —(CH₂)—NH—CO—R^{a}, —(CH₂)ₙ—CO—NH—R^{a}, (CH₂)ₙ—CO—N(R^{a})₂, C₅₋₈ alkyl, C₂₋₄ alkenyl, C₃₋₆ cycloalkyl, C₃₋₆ cycloalkyl-C₁₋₃ alkyl, benzyl, C₃₋₄ heterocyclyl, and C₅₋₇ heterocycloalkyl. In this embodiment, R¹ is more preferably selected from —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, C₅ alkyl, benzyl, C₃₋₅ cycloalkyl-C₁₋₃ alkyl, C₃₋₄ heterocyclyl and C₅ heterocycloalkyl. Preferred substituents for said alkyl include groups independently selected from halogen, hydroxyl, C₁₋₄ alkylthio, N(R^{b})₂, benzyl and methoxy. Preferred substituents for said cycloalkyl, phenyl, benzyl, heterocyclyl or heterocycloalkyl include groups independently selected from halogen, hydroxy, C₁₋₄ alkyl, trifluoromethyl, N(R^{b})₂, methoxy, haloC₁₋₄alkyl, dihaloC₁₋₄alkyl, trihaloC₁₋₄alkyl, halomethoxy, dihalomethoxy, and trihalomethoxy.

For compounds of formula (Ib), in another embodiment, R¹ is preferably C₁₋₄ alkyl substituted with substituted with one group independently selected from halogen, hydroxy, C₁₋₄ alkylthio, N(R^{b})₂, and methoxy, and optionally substituted with 1 or 2 additional groups each independently selected from halogen, hydroxy, C₁₋₄ alkylthio, N(R^{b})₂, phenyl and methoxy.

For compounds of formula (Ib), in a further embodiment, R¹ is preferably phenyl or C₅₋₇ heteroaryl, said phenyl or C₅₋₇ heteroaryl group being substituted with one group independently selected from chlorine, bromine, iodine, and C₁₋₄ alkyl, and optionally substituted with 1 or 2 additional groups each independently selected from halogen, hydroxy, C₁₋₄ alkyl, N(R^{b})₂ and methoxy.

For compounds of formula (Ic), R¹ is preferably selected from C₁₋₄ alkyl, phenyl and C₅₋₇ heteroaryl, said alkyl groups optionally being substituted with 1 or 2 phenyl groups; said phenyl or heteroaryl groups optionally being substituted with 1 or 2 groups independently selected from fluorine, hydroxy, methoxy and N(R^{b})₂.

Preferably, R^{a} is selected from C₁₋₄ alkyl and phenyl. Preferred substituents for said C₁₋₄ alkyl include groups independently selected from C₁₋₄ alkyl, and halogen.

Preferably n is 0, 1 or 2. More preferably n is 0 or 1.

Preferably, R² is independently selected from halogen, C₁₋₄ alkoxy, C₁₋₄ alkyl, and N(R^{b})₂. More preferably, R² is selected from halogen and C₁₋₄ alkyl. Preferred substituents for said alkyl or alkoxy include groups independently selected from halogen, hydroxy, halomethoxy, dihalomethoxy, and trihalomethoxy.

Preferably, R^{b} is selected from hydrogen and C₁₋₄ alkyl. Preferred substituents for said C₁₋₄ alkyl include groups independently selected from C₁₋₄ alkyl.

Preferably m is 0 or 1. More preferably m is 0.

R³ and R⁴ are preferably independently selected from halogen, C₁₋₄ alkyl, fluoromethyl, difluoromethyl and trifluoromethyl. More preferably, R³ and R⁴ are independently selected from halogen and methyl. Amongst the halogens, there are preferred chlorine, bromine, and fluorine, especially chlorine and bromine, in particular bromine.

Preferably, Y is selected from oxygen and methylene. Most preferably, Y is oxygen.

For compounds of formula (I), W is preferably selected from C₁₋₃ alkylene, C₂₋₃ alkenylene, C₂₋₃ alkynylene, N(R^{c})—C₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene and NHC(O)—C₀₋₃ alkylene, said alkylene, alkenylene or alkynylene groups or portions of groups optionally being substituted with 1 or 2 groups selected from hydroxy, mercapto, amino, halo, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy.

For compounds of formula (I), W is more preferably selected from C₁₋₃ alkylene, C₂₋₃ alkenylene, N(R^{c})—C₁₋₂ alkylene, O—C₁₋₂ alkylene, C(O)NH—C₁₋₂ alkylene and NHC(O)—C₁₋₂ alkylene, said alkylene or alkenylene groups or portions of groups optionally being substituted with a group selected from halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy. Preferred halo groups are chloro or fluoro, particularly fluoro. Most preferably, for compounds of formula (I), W is selected from C₁₋₃ alkylene, O—C₁₋₂ alkylene, C(O)NH—C₁₋₂ alkylene and NHC(O)—C₁₋₂ alkylene. Most particularly preferably, for compounds of formula (I), W is ethylene, O—C₁₋₂ alkylene, or C(O)NH—CH₂—. Preferably the alkylene group (for example the ethylene group) is substituted with one or more halo groups, for example one or more fluoro groups (for example one fluoro group). Monohalo C₁₋₃ alkylene (for example fluoro C₁₋₃ alkylene) thus constitutes a preferred group W.

In another preferred embodiment, for compounds of formula (I), W is selected from C₁₋₃ alkylene, C₂₋₃ alkenylene, O—C₁₋₃ alkylene, C(O)NH—C₁₋₂ alkylene and NHC(O)—C₁₋₂ alkylene.

In an alternative preferred embodiment, for compounds of formula (I), W is selected from C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NHC(O)—C₀₋₃ alkylene and C₁₋₃ alkyleneC(O)NH—C₁₋₃ alkylene, said alkylene, alkenylene or alkynylene groups being straight chain, and said alkylene, alkenylene or alkynylene groups or portions of groups optionally being substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms; and straight chain C₁₋₃ alkylene substituted with 1 or 2 halo groups.

For compounds of formula (Ia), in one embodiment, W is preferably selected from C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene and NHC(O)—C₀₋₃ alkylene, said alkylene, alkenylene or alkynylene groups or portions of groups optionally being substituted with 1 or 2 groups selected from hydroxy, mercapto, amino, halo, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy. In this embodiment, W is more preferably selected from C₂₋₃ alkenylene, N(R^{c})—C₁₋₂ alkylene, O—C₁₋₂ alkylene, C(O)NH—C₁₋₂ alkylene and NHC(O)—C₁₋₂ alkylene, said alkylene or alkenylene groups or portions of groups optionally being substituted with a group selected from halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy. Preferred halo groups are chloro or fluoro, particularly fluoro. Most preferably, in this embodiment, W is selected from O—C₁₋₂ alkylene, C(O)NH—C₁₋₂ alkylene and NHC(O)—C₁₋₂ alkylene. Most particularly preferably, W is O—C₁₋₂ alkylene or C(O)NH—CH₂—.

In another embodiment, for compounds of formula (Ia), W is preferably C₁ alkylene substituted with 1 or 2 groups selected from hydroxy, mercapto, amino, halo, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy. More preferably, the C₁ alkylene group is substituted with 1 or 2 groups selected from halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy. Most preferably, the C₁ alkylene group is substituted with 1 or 2 halo groups, for example one or more fluoro groups (for example one fluoro group).

In a further embodiment, for compounds of formula (Ia), W is C₂₋₃ alkylene substituted with 1 or 2 groups selected from mercapto, halo, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy. More preferably, the C₂₋₃ alkylene group is substituted with 1 or 2 groups selected from halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy. Most preferably the C₂₋₃ alkylene group (for example the ethylene group) is substituted with one or more halo groups, for example one or more fluoro groups (for example one fluoro group). Monohalo C₂₋₃ alkylene (for example fluoro C₂₋₃ alkylene) thus constitutes a preferred group W.

For compounds of formula (Ib) or (Ic), W is preferably selected from C₁₋₃ alkylene and C₂₋₃ alkylene substituted with a hydroxy or amino group. More preferably, for compounds of formula (Ib) or (Ic), W is C₁₋₃ alkylene, particularly ethylene or propylene.

R^{c} is preferably selected from hydrogen, C₁₋₂ alkyl, fluoromethyl, difluoromethyl and trifluoromethyl;

R⁵ is preferably selected from —CO₂R^{d}, —PO(OR^{d})₂, —SO₂OR^{d}, —NHSO₂R^{d}, —COCO₂R^{d} and CONR^{d}OR^{d}. More preferably, R⁵ is —CO₂R^{d}, —PO(OR^{d})₂ or —SO₂OR^{d}. Most preferably, R⁵ is —CO₂R^{d}, particularly —CO₂H.

R^{d} is preferably selected from hydrogen, ethyl, methyl, phenyl and phenyl substituted with 1, 2 or 3 groups independently selected from amino, hydroxyl, halogen and methyl, particularly hydrogen.

It is to be understood that features of an embodiment of the invention described with reference to one parameter can be combined with the features of another embodiment. The disclosure herein thus includes the combination of the features of any one embodiment with the features of any other embodiment described. Unless otherwise stated, where structurally appropriate all embodiments and preferred features of compounds of formula (I) apply to compounds of formula (Ia), (Ib) and (Ic).

Compounds according to the invention include:
3-(3,5-dibromo-4-{[2-(3-methoxyphenyl)-1H-benzimidazol-5-yl]oxy}phenyl)propanoic acid
3-{3,5-dibromo-4-[(2-methyl-1H-benzimidazol-5-yl)oxy]phenyl}propanoic acid
{3,5-dibronio-4-[(2-isobutyl-1H-benzimidazol-5-yl)oxy]phenoxy}acetic acid
4-(3,5-dibromo-4-{[2-(4-methylphenyl)-1H-benzimidazol-5-yl]oxy}phenyl)butanoic acid
4-(3,5-dibromo-4-{[2-(4-fluorophenyl)-1H-benzimidazol-5-yl]oxy}phenyl)butanoic acid
4-(3,5-dibromo-4-{[2-(4-Chlorophenyl)-1H-benzimidazol-5-yl]oxy}phenyl)butanoic acid
4-(3,5-dibromo-4-{[2-(3-Chlorophenyl)-1H-benzimidazol-5-yl]oxy}phenyl)butanoic acid
3-(3,5-dibromo-4-{[2-(3,4-dimethylphenyl)-1H-benzimidazol-5-yl]oxy}phenyl)propanoic acid
3-(3,5-dibromo-4-{[2-(3-fluoro-4-methylphenyl)-1H-benzimidazo 1-5 -yl]oxy}phenyl)propanoic acid
3-(3,5-dibromo-4-{[2-isobutyl-1H-benzimidazol-5-yl]oxy}phenyl)-2-fluoropropanoic acid
3-(3,5-dibromo-4-{[2-(3-methylphenyl)-1H-benzimidazol-5-yl]oxy}phenyl)propanoic acid
3-(3,5-dibromo-4-{[2-(3-methyl-4-fluorophenyl)-1H-benzimidazol-5-yl]oxy}phenyl)-2-fluoropropanoic acid
3-(3,5-dibromo-4-{[2-(4-methylphenyl)-1H-benzimidazol-5-yl]oxy}phenyl)-2-fluoropropanoic acid
{3-(3,5-dibromo-4-{[2-(3-methyl-4-fluorophenyl)-1H-benzimidazol-5-yl]oxy}phenoxy}acetic acid
{3,5-dibromo-4-{[2-(2,2-dimethylpropyl)-1H-benzimidazol-5-yl)oxy]phenoxy}acetic acid
{3,5-dibromo-4-[2-(cyclopropylmethyl)-1H-benzimidazol-5-yl)oxy]phenoxy}acetic acid
{3,5-dibromo-4-[2-propyl-1H-benzimidazol-5-yl)oxy]phenoxy}acetic acid
3-(3,5-dichloro4-{[2-(3-methylphenyl)-1H-benzimidazol-5-yl]oxy}phenyl)propanoic acid
3-(3,5-dibromo4-{[2-(2-(methylthio)-ethyl)-1H-benzimidazol-5-yl]oxy}phenyl)propanoic acid
3-(3,5-dibromo-4-{[2-(2-(methylthio)-ethyl)-1H-benzimidazol-5-yl]oxy}phenyl)-2-fluoropropanoic acid
N-(3,5-dibromo4-{[2-2-(methylthio)-ethyl)-1H-benzimidazol-5-yl]oxy}benzoyl)glycine
3-(3,5-dibromo-4-{[2-isopropylcarbamoyl-1H-benzimidazol-5-yl]oxy}phenyl)-2-fluoropropanoic acid
3-(3,5-dibromo-4-{[2-ethylcarbamoyl-1H-benzimidazol-5-yl]oxy}phenyl)-2-fluoropropanoic acid
3-(3,5-dibromo-4-{[2-diisopropylcarbamoyl-1H-benzimidazol-5-yl]oxy}phenyl)-2-fluoropropanoic acid
5-[2,6-Dibromo-4-(2-fluoro-2-isopropylcarbamoyl-ethyl)-phenoxy]-1H-benzoimidazole-2-carboxylic acid
3-{3,5-Dibromo-4-[2-(methylsulfonylamino-methyl)-1H-benzoimidazol-5-yloxy]-phenyl}-propionic acid
3-{3,5-Dibromo-4-[2-(3-fluoro-4-methyl-phenyl)-1H-benzoimidazol-5-yloxy]-phenyl}-2-fluoropropionic acid
[3,5-Dibromo-4-(2-cyclopentylmethyl-1H-benzoimidazol-5-yloxy)-phenoxy]-acetic acid
3-{3,5-Dibromo-4-[2-(1,1,2,2-tetrafluoro-ethyl)-1H-benzoimidazol-5-yloxy]-phenyl}-2-fluoropropionic acid
{3,5-Dibromo-4-[2-(1,1,2,2-tetrafluoro-ethyl)-1H-benzoimidazol-5-yloxy]-phenoxy}-acetic acid
{3,5-Dibromo-4-[2-(1,1,2,2-tetrafluoro-ethyl)-1H-benzoimidazol-5-yloxy)-benzoylamino}-acetic acid
{3,5-Dibromo-4-[2-(1,1,2,2-tetrafluoro-ethyl)-1H-benzoimidazol-5-yloxy]-benzoylamino}-acetic acid methyl ester
3-{3,5-Dibromo-4-[2-(2,5-dimethyl-oxazol4-yl)-1H-benzoimidazol-5-yloxy]-phenyl}-2-fluoropropionic acid
{3,5-Dibromo-4-[2-(2,5-dimethyl-oxazol-4-yl)-1H-benzoimidazol-5-yloxy]-benzoylamino}-acetic acid
{3,5-Dibromo-4-[2-(2,5-dimethyl-oxazol-4-yl)-1H-benzoimidazol-5-yloxy]-phenoxy}-acetic acid
{3,5-Dichloro-4-[2-(1,1,2,2-tetrafluoro-ethyl)-1H-benzoimidazol-5-yloxy]-benzoylamino}-acetic acid
{3,5-Dichloro-4-[2-(2,5-dimethyl-oxazol-4-yl)-1H-benzoimidazol-5-yloxy]-benzoylamino}-acetic acid
[3,5-Dichloro-4-(2-furan-2-yl-1H-benzoimidazol-5-yloxy)-benzoylamino]-acetic acid
{3,5-Dichloro-4-[2-(2-fluoro-phenyl)-1H-benzoimidazol-5-yloxy]-benzoylamino}-acetic acid
{3,5-Dichloro-4-[2-(2-methoxy-phenyl)-1H-benzoimidazol-5-yloxy]-benzoylamino}-acetic acid
{3,5-Dichloro-4-[2-(1-methyl-1H-pyrrol-2-yl)-1H-benzoimidazol-5-yloxy]-benzoylamino}-acetic acid
N-[3,5-Dibromo-4-(2-isobutyl-1H-benzoimidazol-5-yloxy)-phenyl]-acetamide
N-[3,5-Dibromo-4-(2-isobutyl-1H-benzoimidazol-5-yloxy)-phenyl]-malonamic acid methyl ester
3-[3,5-Dichloro-4-(2-isobutyl-1H-benzoimidazol-5-yloxy)-phenyl]-2-fluoro-propionic acid
{3,5-Dibromo-4-[2-(2,5-dimethyl-oxazol-4-yl)-1H-benzoimidazol-5-yloxy]-phenoxy}-acetic acid methyl ester
N-[3,5-Dibromo-4-(2-isobutyl-1H-benzoimidazol-5-yloxy)-phenyl]-malonamic acid
(R)-3-[3,5-Dibromo-4-(2-isobutyl-1H-benzoimidazol-5-yloxy)-phenyl]-2-fluoro-propionic acid
(S)-3-[3,5-Dibromo-4-(2-isobutyl-1H-benzoimidazol-5-yloxy)-phenyl]-2-fluoro-propionic acid
3-[3,5-Dibromo-4-(2-methanesulfonylamino-1H-benzoimidazol-5-yloxy)-phenyl]-2-fluoro-propionic acid
3-[3-Chloro-4-(2-isobutyl-1H-benzoimidazol-5-yloxy)-5-trifluoromethyl-phenyl]-2-fluoro-propionic acid
3-[3,5-Dibromo-4-(2-methylsulfamoyl-1H-benzoimidazol-5-yloxy)-phenyl]-2-fluoro-propionic acid
3-[3,5-Dibromo-4-(2-oxazol-2-yl-1H-benzoimidazol-5-yloxy)-phenyl]-2-fluoro-propionic acid
3-[3,5-Dibromo-4-(2-oxazol-4-yl-1H-benzoimidazol-5-yloxy)-phenyl]-2-fluoro-propionic acid
3-[3;5-Dibromo-4-(2-dimethylamino-1H-benzoimidazol-5-yloxy)-phenyl]-2-fluoro-propionic acid
N-[4-(2-Isobutyl-1H-benzoimidazol-5-yloxy)-3,5-bis-trifluoromethyl-phenyl]-malonamic acid

The compounds names given above were generated in accordance with IUPAC by the ACD Labs 8.0/name program, version 8.05 and/or with ISIS DRAW Autonom 2000.

Preferred compounds according to the invention include:
3-3,5-Dibromo-4-[2-(1,1,2,2-tetrafluoro-ethyl)-1H-benzoimidazol-5-yloxy]-phenyl}-2-fluoro-propionic acid
3-[3,5-Dibromo-4-(2-isobutyl-1H-benzoimidazol-5-yloxy)-phenyl]-2-fluoro-propionic acid
3-{3,5-Dibromo-4-[2-(2,5-dimethyl-oxazol-4-yl)-1H-benzoimidazol-5-yloxy]-phenyl}-2-fluoro-propionic acid
{3,5-Dibromo-4-[2-(1,1,2,2-tetrafluoro-ethyl)-1H-benzoimidazol-5-yloxy]-phenoxy}-acetic acid

Salts and solvates of compounds of formula (I) which are suitable for use in medicine are those wherein a counterion or associated solvent is pharmaceutically acceptable. However, salts and solvates having non-pharmaceutically acceptable counterions or associated solvents are within the scope of the present invention, for example, for use as intermediates in the preparation of the compounds of formula (I) and their pharmaceutically acceptable salts, solvates and physiologically functional derivatives. By the term “physiologically functional derivative” is meant a chemical derivative of a compound of formula (I) having the same physiological function as the free compound of formula (I), for example, by being convertible in the body thereto. According to the present invention, examples of physiologically functional derivatives include esters, amides, and carbamates; preferably esters and amides.

Suitable salts according to the invention include those formed with organic or inorganic acids or bases. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycollic, lactic, salicylic, oxaloacetic, methanesulfonic, ethanesulfonic, p-toluenesulfonic, formic, benzoic, malonic, naphthalene-2-sulfonic, benzenesulfonic, and isethionic acids. Other acids such as oxalic, while not in themselves pharmaceutically acceptable, may be useful as intermediates in obtaining the compounds of the invention and their pharmaceutical acceptable acid addition salts. Pharmaceutically acceptable base salts include ammonium salts, alkali metal salts, for example those of potassium and sodium, alkaline earth metal salts, for example those of calcium and magnesium, and salts with organic bases, for example dicyclohexylamine and N-methyl-D-glucomine.

Pharmaceutically acceptable esters and amides of the compounds of formula (I) may have an appropriate group in the formula, for example an acid group, converted to a C₁₋₆ alkyl, C₅₋₁₀ aryl, C₅₋₁₀aryl-C₁₋₆ alkyl ester or amide. Pharmaceutically acceptable esters of the compounds of formula (I) may have an appropriate group in the formula, for example a hydroxy group, converted to a C₁₋₆ alkyl, C₅₋₁₀ aryl, or C₅₋₁₀ aryl-C₁₋₆ alkyl ester. Pharmaceutically acceptable amides and carbamates of the compounds of formula (I) may have an appropriate group in the formula, for example an amino group, converted to a C₁₋₆ alkyl, C₅₋₁₀ aryl, C₅₋₁₀ aryl-C₁₋₆ alkyl amide, or carbamate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

A compound which, upon administration to the recipient, is capable of being converted into a compound of formula (I) as described above or an active metabolite or residue thereof, is known as a “prodrug”. A prodrug may, for example, be converted within the body, e. g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); and in Edward B. Roche, ed., Bioreversible Carriers in Drug Design, American Pharmaceutical Association and Pergamon Press, 1987, both of which are incorporated herein by reference.

As used herein, the term “alkyl” means.both straight and branched chain saturated hydrocarbon groups. Examples of alkyl groups include methyl, ethyl, n-propyl, iso-propyl, n-butyl, t-butyl, i-butyl, sec-butyl, pentyl, hexyl, heptyl, octyl, nonyl, and decyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl, 1-ethylbutyl, and 1-ethylpentyl groups.

As used herein, the term “alkoxy” means the group O-alkyl, where “alkyl” is used as described above. Examples of alkoxy groups include methoxy and ethoxy groups. Other examples include propoxy and butoxy.

As used herein, the term “alkenyl” means both straight and branched chain unsaturated hydrocarbon groups with at least one carbon carbon double bond. Up to 5 carbon carbon double bonds may, for example, be present. Examples of alkenyl groups include ethenyl, propenyl, butenyl, pentenyl, hexenyl, heptenyl, octenyl, nonenyl, decenyl and dodecenyl. Preferred alkynyl groups include ethenyl, 1-propenyl and 2-propenyl.

As used herein, the term “alkynyl” means both straight and branched chain unsaturated hydrocarbon groups with at least one carbon carbon triple bond. Up to 5 carbon carbon triple bonds may, for example, be present. Examples of alkynyl groups include ethynyl, propynyl, butynyl, pentynyl, hexynyl, heptynyl, octynyl, nonynyl, decynyl and dodecynyl. Preferred alkenyl groups include ethynyl 1-propynyl and 2-propynyl.

As used herein, the term “cycloalkyl” means a saturated group in a ring system. The cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo [2.2.1]hept-2-yl. Preferably, the cycloalkyl group is monocyclic.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aromatic group, one of the rings may, for example, be partially saturated. Examples of such groups include indanyl and tetrahydronaphthyl. Specifically, the term C₅₋₁₀ aryl is used herein to mean a group comprising from 5 to 10 carbon atoms in a monocyclic or bicyclic aromatic group. A particularly preferred C₅₋₁₀ aryl group is phenyl.

As used herein, the term “halogen” means fluorine, chlorine, bromine or iodine. Fluorine, chlorine and bromine are particularly preferred. In some embodiments, fluorine is especially preferred. In alternative embodiments, chlorine or bromine are especially preferred.

As used herein, the term “heterocyclyl” means an aromatic (“heteroaryl”) or a non-aromatic (“heterocycloalkyl”) cyclic group of carbon atoms wherein from one to three of the carbon atoms is/are replaced by one or more heteroatoms independently selected from nitrogen, oxygen or sulfur. A heterocyclyl group may, for example, be monocyclic or bicyclic. In a bicyclic heterocyclyl group there may be one or more heteroatoms in each ring, or only in one of the rings. A heteroatom is preferably O or N. Heterocyclyl groups containing a suitable nitrogen atom include the corresponding N-oxides. Examples of monocyclic heterocycloalkyl rings include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of bicyclic heterocyclic rings in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of bicyclic heteroaryl groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole.

Examples of preferred heterocyclyl groups include piperidinyl, tetrahydrofuranyl, oxazolyl, tetrahydropyranyl, pyridyl, pyrimidyl and indolyl.

As used herein the term “cycloalkylalkyl” means a group cycloalkyl-alkyl- attached through the alkyl group, “cycloalkyl” and “alkyl” being understood to have the meanings outlined above.

As mentioned above, the compounds of the invention have activity as thyroid receptor ligands. The compounds of the invention are agonists or partial agonists of the thyroid receptor. Preferred compounds of the invention are agonists of the thyroid receptor. Compounds of the present invention possess activity as agonists of the thyroid receptor. They may thus be used in the treatment of diseases or disorders associated with thyroid receptor activity, particularly diseases or disorders for which selective agonists of the thyroid receptor-beta are indicated. In particular, compounds of the present invention may be used in the treatment of diseases or disorders associated with metabolism dysfunction or which are dependent upon the expression of a T₃ regulated gene.

The example compounds below are agonists or partial agonists of the thyroid receptor, and exhibit no antagonism of the thyroid receptor.

Clinical conditions for which an agonist or partial agonist is indicated include, but are not limited to, hypothyroidism; subclinical hyperthyroidism; non-toxic goiter; atherosclerosis; thyroid hormone replacement therapy (e.g., in the elderly); malignant tumor cells containing the thyroid receptor; papillary or follicular cancer; maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); accelerating healing of complicated fractures (e.g. distraction osteogenesis); in joint replacement; eating disorders (e.g., anorexia); treatment of obesity and growth retardation associated with obesity; treatment of depression, nervousness, irritability and stress; treatment of reduced mental energy and low self-esteem (e.g., motivatioon/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of hyperinsulinemia; stimulation of osteoblasts, bone remodeling and cartilage growth; regulation of food intake; treatment of insulin resistance, including NIDDM, in mammals (e.g., humans); treatment of insulin resistance in the heart; treatment of congestive heart failure; treatment of musculoskeletal impairment (e.g., in the elderly); improvement of the overall pulmonary function; skin disorders or diseases, such as dermal atrophy, glucocorticoid induced dermal atrophy, including restoration of dermal atrophy induced by topical glucocorticoids, and the prevention of dermal atrophy induced by topical glucocorticoids (such as the simultaneous treatment with topical glucocorticoid or a pharmacological product including both glucocorticoid and a compound of the invention), the restoration/prevention of dermal atrophy induced by systemic treatment with glucocorticoids, restoration/prevention of atrophy in the respiratory system induced by local treatment with glucocorticoids, UV-induced dermal atrophy, dermal atrophy induced by aging (wrinkles, etc.), wound healing, post surgical bruising caused by laser resurfacing, keloids, stria, cellulite, roughened skin, actinic skin damage, lichen planus, ichtyosis, acne, psoriasis, Dernier's disease, eczema, atopic dermatitis, chloracne, pityriasis and skin scarring. In addition, the conditions, diseases, and maladies collectively referenced to as “Syndrome X” or Metabolic 20 Syndrome as detailed in Johannsson J. Clin. Endocrinol. Metab., 82, 727-34 (1997), may be treated employing the compounds of the invention. The term treatment includes, where appropriate, prophylactic treatment.

Accordingly, the compounds of the invention find application in the treatment or prophylaxis of the following: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes; (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

The compounds of the invention find particular application in the treatment or prophylaxis of the following: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes; (7) depression; (8).goiter; (9) thyroid cancer; and (10) glaucoma.

The compounds of the invention find especial application in the treatment or prophylaxis of the following: (1) hypercholesterolemia, dyslipidemia or any other lipid disorder manifested by an unbalance of blood or tissue lipid levels; (2) atherosclerosis; (3) obesity; (4) diabetes.

The invention also provides a method for the treatment or prophylaxis of a condition that may be treated with a thyroid receptor agonist or partial agonist in a mammal, which comprises administering to the mammal a therapeutically effective amount of a compound of formula (I) as defined above or a pharmaceutically acceptable ester, amide, solvate or salt thereof, including a salt of such an ester or amide, and a solvate of such an ester, amide or salt. Clinical conditions mediated by a thyroid receptor that may be treated with a thyroid receptor agonist or partial agonist are those described above.

The invention also provides a compound of formula (I) as defined above or a pharmaceutically acceptable ester, amide, solvate or salt thereof, including a salt of such an ester or amide, and a solvate of such an ester, amide or salt, for use in the treatment or prophylaxis of a condition that may be treated with a thyroid receptor agonist or partial agonist. The invention also provides a compound of formula (Ia), (Ib) or (Ic) as defined above or a pharmaceutically acceptable ester, amide, solvate or salt thereof, including a salt of such an ester or amide, and a solvate of such an ester, amide or salt, for use as a medicament; in particular, for use in the treatment or prophylaxis of a condition that may be treated with a thyroid receptor agonist or partial agonist. Clinical conditions mediated by a thyroid receptor that may be treated with a thyroid receptor agonist or partial agonist are those described above.

Hereinafter, the term “active ingredient” means a compound of formula (I), (Ia), (Ib) or (Ic) as defined above, or a pharmaceutically acceptable ester, amide, solvate or salt thereof, including a salt of such an ester or amide, and a solvate of such an ester, amide or salt. Compounds of formula (I), (Ia), (Ib) or (Ic) are referred to herein as compounds of the invention.

The amount of active ingredient which is required to achieve a therapeutic effect will, of course, vary with the particular compound, the route of administration, the subject under treatment, and the particular disorder or disease being treated. The compounds of the invention may be administered orally or via injection at a dose of from 0.05 to 500 mg/kg per day, preferably 0.05 to 100 mg/kg per day. The dose range for adult humans is generally from 5 mg to 35 g per day and preferably 5 mg to 2 g per day. Tablets or other forms of presentation provided in discrete units may conveniently contain an amount of compound of the invention which is effective at such dosage or as a multiple of the same, for example units containing 5 mg to 500 mg, usually around 10 mg to 200 mg.

While it is possible for the active ingredient to be administered alone, it is preferable for it to be present in a pharmaceutical formulation or composition. Accordingly, the invention provides a pharmaceutical formulation comprising a compound of formula (Ia), (Ib) or (Ic) as defined above or a pharmaceutically acceptable ester, amide, solvate or salt thereof, including a salt of such an ester or amide, and a solvate of such an ester, amide or salt, and a pharmaceutically acceptable excipient. Pharmaceutical compositions of the invention may take the form of a pharmaceutical formulation as described below.

The pharmaceutical formulations according to the invention include those suitable for oral, parenteral (including subcutaneous, intradermal, intramuscular, intravenous, and intraarticular), inhalation (including fine particle dusts or mists which may be generated by means of various types of metered does pressurized aerosols), nebulizers or insufflators, rectal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compounds of the invention can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an iotonic sodium chloride solution, or other suitable dispersing or wetting and suspending agents, including synthetic mono- or diglycerides, and fatty acids, including oleic acid, or Cremaphor.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

Whilst a compound of the invention may be used as the sole active ingredient in a medicament, it is also possible for the compound to be used in combination with one or more further active agents. Such further active agents may be further compounds according to the invention, or they may be different therapeutic agents, for example an anti-dyslipidemic agent or other pharmaceutically active material.

The compounds of the present invention may be employed in combination with one or more other modulators and/or ligands of the thyroid receptor or one or more other suitable therapeutic agents selected from the group consisting of cholesterol/lipid lowering agents, hypolipidemic agents, anti-atherosclerotic agents, anti-diabetic agents, anti-osteoporosis agents, anti-obesity agents, growth promoting agents, anti-inflammatory agents, anti-anxiety agents, anti-depressants, anti-hypertensive agents, cardiac glycosides, appetite suppressants, bone resorption inhibitors, thyroid mimetics, anabolic agents, anti-tumor agents and retinoids.

Examples of suitable hypolipidemic agents for use in combination with the compounds of the present invention include an acyl coenzyme A cholesterol acyltransferase (ACAT) inhibitor, a microsomal triglyceride transfer protein (MTP) inhibitor, a cholesterol ester transfer protein (CETP) inhibitor, a ileal bile acid transporter (IBAT) inhibitor, any cholesterol absorption inhibitor, a 3-hydroxy-3-methylglutaryl coenzyme A (HMG-CoA) reductase inhibitor, a squalene synthetase inhibitor, a bile acid sequestrant, a peroxisome proliferator-activator receptor (PPAR)-alpha agonist, a peroxisome proliferator-activator receptor (PPAR)-delta agonist, any peroxisome proliferator-activator receptor (PPAR)-gamma/delta dual agonist, any peroxisome proliferator-activator receptor (PPAR)-alphaldelta dual agonist, a nicotinic acid or a derivative thereof, and a thiazolidinedione or a derivative thereof.

Examples of suitable hypolipidemic agents for use in combination with the compounds of the present invention also include ezetimibe, simvastatin, atorvastatin, rosuvastatin, cerivastatin, fluvastatin, lovastatin, pravastatin, fenofibrate, gemfibrozil and bezafibrate.

Examples of suitable anti-diabetic agents for use in combination with the compounds of the present invention include biguanides (e.g., metformin or phenformin), glucosidase inhibitors (e.g., acarbose or miglitol), insulins (including insulin secretagogues or insulin sensitizers), meglitinides (e.g., repaglinide), sulfonylureas (e.g., glimepiride, glyburide, glipyride, gliclazide, chlorpropamide and glipizide), biguanide/glyburide combinations (e.g., Glucovance®), thiazolidinediones (e.g., troglitazone, rosiglitazone, englitazone, darglitazone and pioglitazone), PPAR-alpha agonists, PPAR-gamma agonists, PPAR alpha/gamma dual agonists, PPAR alpha/delta dual agonists, SGLT 1, 2 or 3 inhibitors, glycogen phosphorylase inhibitors, inhibitors of fatty acid binding protein (aP2), glucagon-like peptide-1 (GLP-1), glucocorticoid (GR) antagonist and dipeptidyl peptidase IV (DP4) inhibitors.

Examples of suitable anti-osteoporosis agents for use in combination with the compounds of the present invention include alendronate, risedronate, PTH, PTH fragment, raloxifene, calcitonin, RANK ligand antagonists, calcium sensing receptor antagonists, TRAP inhibitors, selective estrogen receptor modulators (SERM) and AP-1 inhibitors.

Examples of suitable anti-obesity agents for use in combination with the compounds of the present invention include aP2 inhibitors, PPAR gamma antagonists, PPAR delta agonists, beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), other thyroid receptor beta drugs, such as a thyroid receptor ligand as disclosed in WO 97/21993 (U. Cal SF), WO 99/00353 (KaroBio) and WO00/39077 (KaroBio), CB-1 (cannabinoid receptor) antagonists (see G. Colombo et al, “Appetite Suppression and Weight Loss After the Cannabinoid Antagonist SR 141716”, Life Sciences, Vol 63, PL 113-117 (1998)) and/or an anorectic agent, such as dexamphetamine, phentermine, phenylpropanolamine or mazindol.

The compounds of the present invention may be combined with growth promoting agents, such as, but not limited to, TRH, diethylstilbesterol, theophylline, enkephalins, E series prostaglandins, compounds disclosed in U.S. Pat. No. 3,239,345, e.g., zeranol, and compounds disclosed in U.S. Pat. No. 4,036,979, e.g., sulbenox or peptides disclosed in U.S. Pat. No. 4,411,890.

The compounds of the invention may also be used in combination with growth hormone secretagogues such as GHRP-6, GHRP-1 (as described in U.S. Pat. No. 4,411,890 and publications WO 89/07110 and WO 89/07111), GHRP-2 (as described in WO 93/04081), NN703 (Novo Nordisk), LY444711 (Lilly), MK-677 (Merck), CP424391 (Pfizer) and B-HT920, or with growth hormone releasing factor and its analogs or growth hormone and its analogs or somatomedins including IGF-1 and IGF-2, or with alpha-adrenergic agonists, such as clonidine or serotinin 5-HT_{D} agonists, such as sumatriptan, or agents which inhibit somatostatin or its release, such as physostigmine and pyridostigmine. A still further use of the disclosed compounds of the invention is in combination with parathyroid hormone, PTH(1-34) or bisphosphonates, such as MK-217 (alendronate).

Examples of suitable anti-inflammatory agents for use in combination with the compounds of the present invention include prednisone, dexamethasone, Enbrel®, cyclooxygenase inhibitors (i.e., COX-1 and/or COX-2 inhibitors such as NSAIDs, aspirin, indomethacin, ibuprofen, piroxicam, Naproxen®, Celebrex®, Vioxx®), CTLA4-Ig agonists/antagonists, CD40 ligand antagonists, IMPDH inhibitors, such as mycophenolate (CellCept®), integrin antagonists, alpha-4beta-7 integrin antagonists, cell adhesion inhibitors, interferon gamma antagonists, ICAM-1, tumor necrosis factor (TNF) antagonists (e.g., infliximab, OR1384), prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, and therapies for the treatment of irritable bowel syndrome (e.g., Zelmac® and Maxi-K® openers such as those disclosed in U.S. Pat. No. 6,184,231 B1). examples of suitable anti-anxiety agents for use in combination with the compounds of the present invention include diazepam, lorazepam, buspirone, oxazepam, and hydroxyzine pamoate.

Examples of suitable anti-depressants for use in combination with the compounds of the present invention include citalopram, fluoxetine, nefazodone, sertraline, and paroxetine.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable cardiac glycosides for use in combination with the compounds of the present invention include digitalis and ouabain.

Examples of suitable cholesterol/lipid lowering agents for use in combination with the compounds of the present invention include HMG-CoA reductase inhibitors, squalene synthetase inhibitors, fibrates, bile acid sequestrants, ACAT inhibitors, MTP inhibitors, lipooxygenase inhibitors, an ileal Na⁺/bile acid cotransporter inhibitor, cholesterol absorption inhibitors, and cholesterol ester transfer protein inhibitors (e.g., CP-529414).

MTP inhibitors which may be employed herein in combination with one or more compounds of formula (I) include MTP inhibitors as disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440 all incorporated herein by reference.

The HMG CoA reductase inhibitors which may be employed in combination with one or more compounds of formula (I) include mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and elated compounds as disclosed in U.S. Pat. Nos. 4,448,784 and 4,450,171. Further HMG CoA reductase inhibitors which may be employed herein include fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, pyrazole analogs of mevalonolactone derivatives as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No.0,142,146 A2, as well as other known HMG CoA reductase inhibitors.

The squalene synthetase inhibitors which may be used in combination with the compounds of the present invention include, but are not limited to, α-phosphono-sulfonates disclosed in U.S. Pat. No. 5,712,396, those disclosed by Biller et al, J. Med. Chem., 1988, Vol. 31,No. 10, pp 1869-1871, including isoprenoid (phosphinylmethyl)phosphonates, terpenoid pyrophosphates disclosed by P. Ortiz de Montellano et al, J. Med. Chem., 1977, 20 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, as well as other squalene synthetase inhibitors as disclosed in U.S. Pat. No. 4,871,721 and 4,924,024 and in Biller, S. A., Neuenschwander, K., Ponpipom, M. M., and Poulter, C. D., Current Pharmaceutical Design, 2, 1-40 (1996).

Bile acid sequestrants which may be used in combination with the compounds of the present invention include cholestyramine, colestipol and DEAE-Sephadex (Secholex®, Policexide®), as well as lipostabil (Rhone-PoulencY, Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride) and ionenes such as disclosed in U.S. Pat. No. 4,027,009, and other known serum cholesterol lowering agents.

ACAT inhibitors suitable for use in combination with compounds of the invention include ACAT inhibitors as described in, Drugs of the Future 24, 9-15 (1999), (Avasimibe); “The ACAT inhibitor, C1-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62.

Examples of suitable cholesterol absorption inhibitor for use in combination with the compounds of the invention include SCH48461 (Schering-Plough), as well as those disclosed in Atherosclerosis 115, 45-63 (1995) and J. Med. Chem. 41, 973 (1998).

Examples of suitable ileal Na⁺/bile acid cotransporter inhibitors for use in combination with the compounds of the invention include compounds as disclosed in Drugs of the Future, 24, 425-430 (1999).

Examples of suitable thyroid mimetics for use in combination with the compounds of the present invention include thyrotropin, polythyroid, KB-130015, and dronedarone.

Examples of suitable anabolic agents for use in combination with the compounds of the present invention include testosterone, TRH diethylstilbesterol, estrogens, β-agonists, theophylline, anabolic steroids, dehydroepiandrosterone, enkephalins, E-series prostaglandins, retinoic acid and compounds as disclosed in U.S. Pat. No. 3,239,345, e.g., Zeranol®; U.S. Pat. No. 4,036,979, e.g., Sulbenox® or peptides as disclosed in U.S. Pat. No. 4,411,890.

For the treatment of skin disorders or diseases as described above, the compounds of the present invention may be used alone or optionally in combination with a retinoid, such as tretinoin, or a vitamin D analog.

A still further use of the compounds of the invention is in combination with estrogen, testosterone, a selective estrogen receptor modulator, such as tamoxifen or raloxifene, or other androgen receptor modulators, such as those disclosed in Edwards, J. P. et al., Bio. Med. Chem. Lett., 9, 1003-1008 (1999) and Hamann, L. G. et al., J. Med. Chem., 42, 210-212 (1999).

A further use of the compounds of this invention is in combination with steroidal or non-steroidal progesterone receptor agonists (“PRA”), such as levonorgestrel, medroxyprogesterone acetate (MPA).

The above other therapeutic agents, when employed in combination with the compounds of the present invention, may be used, for example, in those amounts indicated in the Physicians' Desk Reference (PDR) or as otherwise determined by one of ordinary skill in the art.

Where the compounds of the invention are utilized in combination with one or more other therapeutic agent(s), either concurrently or sequentially, the following combination ratios and dosage ranges are preferred:

When combined with a hypolipidemic agent, an antidepressant, a bone resorption inhibitor and/or an appetite suppressant, the compounds of the invention may be employed in a weight ratio to the additional agent within.the range from about 500:1 to about 0.005:1, preferably from about 300:1 to about 0.01:1.

Where the antidiabetic agent is a biguanide, the compounds of the invention may be employed in a weight ratio to biguanide within the range from about 0.01:1 to about 100:1, preferably from about 0.5:1 to about 2:1.

The compounds of the invention may be employed in a weight ratio to a glucosidase inhibitor within the range from about 0.01:1 to about 100:1, preferably from about 0.5:1 to about 50:1.

The compounds of the invention may be employed in a weight ratio to a sulfonylurea in the range from about 0.01:1 to about 100:1, preferably from about 0.2:1 to about 10:1.

The compounds of the invention may be employed in a weight ratio to a thiazolidinedione in an amount within the range from about 0.01:1 to about 100:1, preferably from about 0.5:1 to about 5:1. The thiazolidinedione may be employed in amounts within the range from about 0.01 to about 2000 mg/day, which may optionally be administered in single or divided doses of one to four times per day. Further, where the sulfonylurea and thiazolidinedione are to be administered orally in an amount of less than about 150 mg, these additional agents may be incorporated into a combined single tablet with a therapeutically effective amount of the compounds of the invention.

Metformin, or salt thereof, may be employed with the compounds of formula (I) in amounts within the range from about 500 to about 2000 mg per day, which may be administered in single or divided doses one to four times daily.

The compounds of the invention may be employed in a weight ratio to a PPAR-alpha agonist, a PPAR-gamma agonist, a PPAR-alpha/gamma dual agonist, an SGLT2 inhibitor and/or an aP2 inhibitor within the range from about 0.01:1 to about 100:1, preferably from about 0.5:1 to about 5:1.

An MTP inhibitor may be administered orally with the compounds of the invention in an amount within the range of from about 0.01 mg/kg to about 100 mg/kg and preferably from about 0.1 mg/kg to about 75 mg/kg, one to four times daily. A preferred oral dosage form, such as tablets or capsules, may contain the MTP inhibitor in an amount of from about 1 to about 500 mg, preferably from about 2 to about 400 mg, and more preferably from about 5 to about 250 mg, administered on a regimen of one to four times daily. For parenteral administration, the MTP inhibitor may be employed in an amount within the range of from about 0.005 mg/kg to about 10 mg/kg and preferably from about 0.005 mg/kg to about 8 mg/kg, administered on a regimen of one to four times daily.

A HMG CoA reductase inhibitor may be administered orally with the compounds of the invention within the range of from about 1 to 2000 mg, and preferably from about 4 to about 200 mg. A preferred oral dosage form, such as tablets or capsules, will contain the HMG CoA reductase inhibitor in an amount from about 0.1 to about 100 mg, preferably from about 5 to about 80 mg, and more preferably from about 10 to about 40 mg.

A squalene synthetase inhibitor may be administered with the compounds of the invention within the range of from about 10 mg to about 2000 mg and preferably from about 25 mg to about 200 mg.

A preferred oral dosage form, such as tablets or capsules, will contain the squalene synthetase inhibitor in an amount of from about 10 to about 500 mg, preferably from about 25 to about 200 mg.

The compounds of the invention as described above also find use, optionally in labelled form, as a diagnostic agent for the diagnosis of conditions associated with malfunction of the thyroid receptor. In particular, the compounds of formula (I) as defined above also find use, optionally in labelled form, as a diagnostic agent for the diagnosis of conditions that may be treated with a thyroid receptor agonist or partial agonist. For example, such a compound may be radioactively labelled.

The compounds of formula (Ia), (Ib) or (Ic) as described above, optionally in labelled form, also find use as a reference compound in methods of discovering other agonists or partial agonists of the thyroid receptor. Thus, the invention provides a method of discovering a ligand of the thyroid receptor which comprises use of a compound of the invention or a compound of the invention in labelled form, as a reference compound. For example, such a method may involve a competitive binding experiment in which binding of a compound of formula (Ia), (Ib) or (Ic) to the thyroid receptor is reduced by the presence of a further compound which has thyroid receptor-binding characteristics, for example stronger thyroid receptor-binding characteristics than the compound of formula (Ia), (Ib) or (Ic) in question.

Numerous synthetic routes to the compounds of the present invention can be devised by any person skilled in the art and the possible synthetic routes described below are not limiting the invention. Many methods exist in the literature for the synthesis of diaryl ethers, for example, two references directly apply to the synthesis of thyroid hormone analogs: Evans D. A. et al. Tetrahedron Lett., 39, 2937-2940, 1998 and Salamonczyk G. M. et al., Tetrahedron Lett., 38, 6965-6968, 1997.

In particular, methods for synthesizing compounds of formula (Ia), (Ib) or (Ic) in which Y is S and CH₂ are generally described in the literature (Y is S: Harrington, C. R., Biochem. J., 43, 434-437, 1948; Dibbo, A. et al., J. Chem. Soc., 2890-2902, 1961; Yokoyama, N. et al., U.S. Pat. No. 5,401,772, 1995; Y is CH₂: Horner, L., Medem, H. H. G., Chem. Ber., 85, 520-530,1952; Chiellini, G. et al., Chemistry & Biology, 5, 299-306, 1998).

The invention also provides a method for preparing a compound of formula (Ia), (Ib) or (Ic) in accordance with the invention as described above wherein Y is oxygen, sulphur or N(R^{b}) comprising a step of reacting
a compound of formula (II)

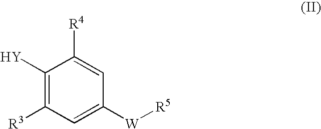

wherein W, R³, R⁴, and R⁵ are as defined above and Y is oxygen, sulphur or N(R^{b})
with a compound of formula (III)

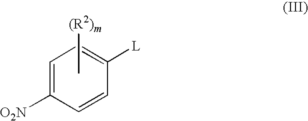

wherein R² and m are as defined above and L is a suitable leaving group, optionally in the presence of a suitable base and, optionally, in the presence of copper powder, followed optionally by interconversion to another compound in accordance with the invention.

Suitable leaving groups L include halogens, for example a fluoride. Suitable bases include carbonates, alkylamines and alkali metal hydroxides, for example potassium carbonate, cesium carbonate, potassium hydroxide, sodium hydroxide, diisopropylamine and triethylamine. Other combinations of leaving groups and bases may be employed, as is known by the person skilled in the art. Optionally, one or more coupling reagents may be employed. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Preferred compounds of formula (11) include:
Methyl 3-(3,5-dibromo-4-hydroxy-phenyl)-propanoate
Methyl (E)-3-(3,5-dibromo-4-hydroxy-phenyl)-acrylate
Methyl (3,5-dibromo-4-hydroxy-phenoxy)-acetate
Methyl 3-(3,5-dibromo-4-hydroxy-phenyl)-2-fluoro-propanoate
Methyl (3,5-dibromo-4-hydroxy-benzoylamino)-acetate
Methyl 3-(3,5-dibromo-4-hydroxy-phenyl)-butanoate
Methyl 3-(3,5-dibromo-4hydroxy-phenyl)2-hydroxy-propanoate
2,6-Dibromo-4-methyl-phenol
2,6-Dichloro-4-methyl-phenol
Methyl 3-(3,5-dichloro-4-hydroxy-phenyl)-propanoate

Preferred compounds of formula (III) include:
1-Fluoro-4-nitro-benzene
2-Chloro-4-fluoro-1-nitro-benzene

The invention also provides an alternative method for preparing a compound of formula (Ia), (Ib) or (Ic) in accordance with the invention as described above wherein Y is oxygen, sulphur, methylene or N(R^{b})

comprising a step of reacting
a compound of formula (IV)

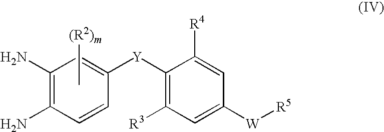

wherein R², R³, R⁴, R⁵, m and W are as defined above and Y is oxygen, sulphur, methylene or N(R^{b})
with a compound of formula (V)

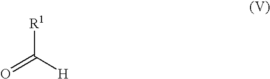

wherein R¹ is as defined above

in the presence of a suitable acid and, followed optionally by interconversion to another compound of formula (Ia), (Ib) or (Ic) wherein Y is oxygen, sulphur, methylene or N(R^{b}).

Suitable acids for use in the reaction include sodium bisulphite.

Other acids may be employed, as is known by the person skilled in the art. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Preferred compounds of formula (IV) include:
Methyl 3-[4-(4,3-diamino-phenoxy)-3,5-dibromo-phenyl]-propanoate
Methyl 3-[4-(4,3-diamino-phenoxy)-3,5-dibromo-phenyl]-2-fluoro-propanoate
Methyl (E)-3-[4-(4,3-diamino-phenoxy)-3,5-dibromo-phenyl]-acrylate
Methyl [4-(4,3-diamino-phenoxy)-3,5-dibromo-phenoxy]-acetate
Methyl [4-(4,3-diamino-phenoxy)-3,5-dibromo-benzoylamino]-acetate

Particularly preferred compounds of formula (IV) include:
Methyl 3-[4-(4,3-diamino-phenoxy)-3,5-dibromo-phenyl]-propanoate
Methyl 3-[4-(4,3-diamino-phenoxy)-3,5-dibromo-phenyl]-2-fluoro-propanoate

Preferred compounds of formula (V) include:
2-Methyl-propionaldehyde
4-Methyl-benzaldehyde
4-Bromo-benzaldehyde
3-Methoxy-benzaldehyde
(2-Oxo-ethyl)-carbamic acid tert-butyl ester

Particularly preferred compounds of formula (V) include:
3-Methoxy-benzaldehyde
(2-Oxo-ethyl)-carbamic acid tert-butyl ester

The invention also provides a method for preparing a compound of formula (Ia), (Ib) or (Ic) in accordance with the invention as described above wherein Y is oxygen, sulphur, methylene or N(R^{b}) comprising a step of reacting
a compound of formula (VI)

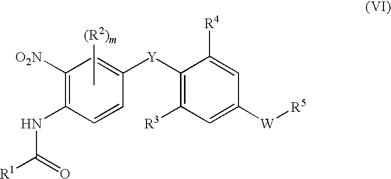

wherein R¹, R², R³, R⁴, R⁵, m and W are as defined above and Y is oxygen, sulphur, methylene or N(R^{b})

with a suitable reducing agent followed by heating in the presence of a suitable acid and, followed optionally by interconversion to another compound of formula (Ia), (Ib) or (Ic) wherein Y is oxygen, sulphur, methylene or N(R^{b}).

Suitable reducing agents include Fe, SnCl₂ and H₂/PtO₂.

Suitable acids for use in the reaction include acetic acid.

Other reducing agents or acids may be employed, as is known by the person skilled in the art. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Preferred compounds of formula (VI) include:
Methyl 3-[4-(4-isobutyrylamino-3-nitrophenoxy)-3,5-dibromo-phenyl]-2-fluoro-propanoate
Methyl 3-{4-[4-(4-methylbenzoyl)amino-3-nitrophenoxy]-3,5-dibromo-phenyl}-2-fluoro-propanoate
Methyl {4-[4-(2,2-dimethylpropionyl)amino-3-nitrophenoxy]-3,5-dibromo-phenoxy}-acetate

The invention also provides a method for preparing a compound of formula (Ia), (Ib) or (Ic) in accordance with the invention as described above wherein Y is oxygen, sulphur or nitrogen comprising a step of reacting
a compound of formula (II)

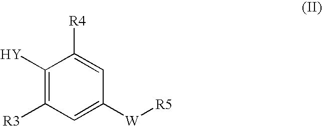

wherein W, R³, R⁴, and R⁵ are as defined above and Y is oxygen, sulphur or N(R^{b}) with a compound of formula (VII)

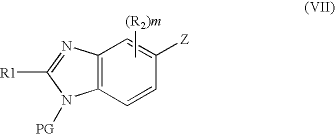

wherein R¹, R² and m are as defined above and PG is a protecting group, and Z is a suitable leaving group, optionally in the presence of a suitable base and optionally, in the presence of copper powder, followed optionally by removal of the protecting group and optionally by interconversion to another compound of the invention.

Suitable leaving groups Z include halogens and boron derivatives, for example a fluoride. Suitable bases include carbonates, alkylamines and alkali metal hydroxides, for example potassium carbonate, cesium carbonate, potassium hydroxide, sodium hydroxide, diisopropylamine and triethylamine. Other combinations of leaving groups and bases may be employed, as is known by the person skilled in the art. Optionally, one or more coupling reagents may be employed. The reaction mixture may be stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The groups Y′ and Z could be switched, being the leaving group in the (II) fragment (the nucleophilic substituent, Z) and the electrophilic radical YH in the (VII) fragment.

Preferred compounds of formula (II) include:
Methyl 3-(3,5-Dibromo-4-hydroxy-phenyl)-propanoate
Methyl (E)-3-(3,5-Dibromo-4-hydroxy-phenyl)-acrylate
Methyl (3,5-Dibromo-4-hydroxy-phenoxy)-acetate
Methyl 3-(3,5-Dibromo-4-hydroxy-phenyl)-2-fluoro-propanoate
Methyl (3,5-Dibromo-4-hydroxy-benzoylamino)-acetate
Methyl 3-(3,5-dibromo4-hydroxy-phenyl)-butanoate
Methyl 3-(3,5-dibromo-4hydroxy-phenyl)2-hydroxy-propanoate
2,6-Dibromo-4-methyl-phenol
2,6-Dichloro-4-methyl-phenol
Methyl 3-(3,5-d ichloro-4-hydroxy-phenyl)-propanoate

The invention also provides a method for preparing a compound of formula (Ia), (Ib) or (Ic) in accordance with the invention as described above wherein Y is methylene comprising a step of reacting
a compound of formula (VIII)

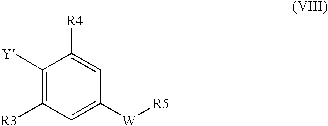

wherein W, R³, R⁴, and R⁵ are as defined above and Y′ is CHO with a compound of formula (IX)

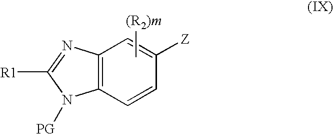

wherein R¹, R² and m are as defined above and PG is a protecting group, and Z may for example be lithium or a Mg-halide, such as MgBr or MgCl. Alternatively, Z maybe a derivative of Sn, Pd, B or Cu.

Other combinations to produce a nucleophilic attack to an aldehyde may be employed, as is known by the person skilled in the art. Optionally, one or more coupling reagents may be employed. The reaction mixture may be stirred at room temperature, cooled or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The groups Y′ and Z could be switched, being the leaving group in the (VIII) fragment (the metal substituent, Z) and the aldehyde in the (IX) fragment.

The invention also provides an alternative method for preparing a compound of formula (Ia), (Ib) or (Ic) in accordance with the invention as described above wherein Y is oxygen, sulphur, methylene or N(R^{b})

comprising a step of reacting
a compound of formula (X)

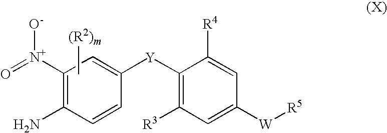

wherein R², R³, R⁴, R⁵, m and W are as defined above and Y is oxygen, sulphur, methylene or N(R^{b})
with a compound of formula (XI)

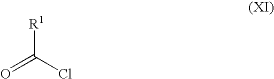

wherein R¹ is as defined above and where the acid chlorine moiety can be substituted with acetic anhydride and other acylating agents known to the person skilled in the art

in the presence of a suitable reducing agent and followed by heating in the presence of a suitable acid and, followed optionally by interconversion to another compound of formula (Ia), (Ib) or (Ic) wherein Y is oxygen, sulphur, methylene or N(R^{b}).

Suitable reducing agents include Fe, SnCl₂ and H₂/PtO₂.

Suitable acids for use in the reaction include acetic acid.

Other acids may be employed, as is known by the person skilled in the art. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Preferred compounds of formula (X) include:
3-[4-(4-Amino-3-nitro-phenoxy)-3,5-dibromo-phenyl]-propionic acid methyl ester
3-[4-(4-Amino-3-nitro-phenoxy)-3,5-dibromo-phenyl]-propionic acid methyl ester
[4-(4-Amino-3-nitro-phenoxy)-3,5-dibromo-benzoylamino]-acetic acid methyl ester
N-[4-(4-Amino-3-nitro-phenoxy)-3,5-dibromo-phenyll-malonamic acid methyl ester
[4-(4-Amino-3-nitro-phenoxy)-3,5-dibromo-phenoxy]-acetic acid methyl ester
[4-(4-Amino-3-nitro-phenoxy)-3,5-dichloro-benzoylamino]-acetic acid methyl ester
3-[4-(4-Amino-3-nitro-phenoxy)-3,5-dichloro-phenyl]-2-fluoro-propionicacid methyl ester
3-[4-(4-Amino-3-nitro-phenoxy)-3-chloro-5-trifluoromethyl-phenyl]-2-fluoro-propionic acid methyl ester

Particularly preferred compounds of formula (X) include:
3-[4-(4-Amino-3-nitro-phenoxy)-3,5-dibromo-phenyl]-propionic acid methyl ester
3-[4-(4-Amino-3-nitro-phenoxy)-3,5-dibromo-phenyl]-propionic acid methyl ester
[4-(4-Amino-3-nitro-phenoxy)-3,5 -dibromo-benzoylamino]-acetic acid methyl ester
N-[4-(4-Amino-3-nitro-phenoxy)-3,5-dibromo-phenyl]-malonamic acid methyl ester
[4-(4-Amino-3-nitro-phenoxy)-3,5-dibromo-phenoxy]-acetic acid methyl ester
[4-(4-Amino-3-nitro-phenoxy)-3,5-dichloro-benzoylamino]-acetic acid methyl ester
3-[4-(4-Amino-3-nitro-phenoxy)-3,5-dichloro-phenyl]-2-fluoro-propionicacid methyl ester

Other suitable conditions and reagents suitable for use in the above reactions for the preparation of compounds of formula (Ia), (Ib) or (Ic) in accordance with the invention or for the synthesis of intermediates suitable for preparing compounds of formula (Ia), (Ib) or (Ic) are described in the following references:
Hauel, R. B., J. Med. Chem., 44, 2001, 1516-1529.
Baudy, M., J. Med. Chem., 47, 2004, 3853-3864.
Yang, D. et al., Synthesis, 2005, 0047-0056.
Louvet, P. et al., Eur. J Med. Chem., 28, 1993, 71-75.
Cowart, M., J. Med. Chem., 47, 2004, 3853-3864.
Garuti, L., II Farmaco, 55, 2000, 35-39.
Gallagher, T., Pardoe, D. A. and Porter, R. A., Tetrahedron Letters, 41, 2000, 5415-5418.

### Examples

The following compounds illustrate compounds of the invention or, where appropriate, compounds for use in the invention.

General Experimental Conditions

Compounds were analyzed on HPLC-MS with alternating ± API and equipped with different brands of 50 mm*2.1 mm, 5μ C8 columns. Elution was with 0.05% formic acid/acetonitrile or 0.05% ammonium acetate/acetonitrile.

MW calculated is an isotopic average and the “found mass” is referring to the most abundant isotope detected in the LC-MS.

### Intermediate 1

### Methyl 3-14-(4-aminophenoxy)-3,5-dibromophenylipropanoate

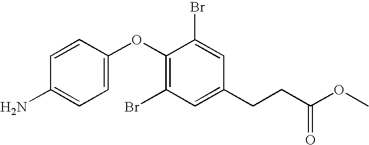

A solution of p-fluoro nitrobenzene (210 mg, 1.5 mmol), methyl 3-(4-hydroxy-3,5-dibromophenyl) propanoate (500 mg, 1.5 mmol) and potassium carbonate (410 mg, 3 mmol) in dimethylsulfoxide (3 mL) was purged with nitrogen and heated at 130° C. for 17 h. The mixture was diluted with ethyl acetate and washed with sodium bicarbonate (sat), water and brine. The combined organic phases were evaporated on silica and purified by flash chromatography (heptane /ethyl acetate 10:0 to 5:5) to give methyl 3-[3,5-dibromo-4-(4-nitrophenoxy)phenyl]propanoate as a white solid (504 mg, yield: 74%).

To a stirred solution of methyl 3-[3,5-dibromo-4-(4-nitrophenoxy)phenyl]propanoate (505 mg, 1.1 mmol) in acetic acid (25 mL) and water (3 mL), iron powder (308 mg, 5.5 mmol) was added. The reaction mixture was stirred for 17 h at 20° C. Acetic acid was removed under vacuum and the residue was diluted with ethyl acetate (50 mL) and water (50 mL) and extracted with ethyl acetate (2×5 mL). The combined ethyl acetate layers were washed with brine, dried over sodium sulphate, filtrated and concentrated. The residue was purified by flash chromatography (dichloromethane/methanol 10:0 to 9:1) to afford the title compound (310 mg) in 72% yield (MW=429.1). LC/MS (ESI): m/z 430.4 (M+1).

### Intermediate 2

### Methyl 4-[4-(4-aminophenoxy)-3,5-dibromophenylIbutanoate

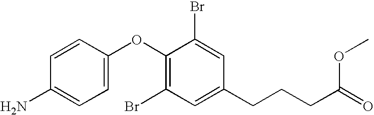

A solution ofp-fluoro-nitrobenzene (282 mg, 2 mmol), methyl 3-(4-hydroxy-3,5-dibromophenyl) butanoate (704 mg, 2 mmol) and potassium carbonate (506 mg, 4 mmol) in dimethylsulfoxide (3 mL) was purged with nitrogen and heated to 130° C. for 17 h. The mixture was diluted with ethyl acetate and washed with sodium bicarbonate (sat), water and brine. The combined organic phases were evaporated on silica and purified by flash chromatography (heptane/ethyl acetate 10:0 to 5:5) to give methyl 3-[3,5-dibromo-4-4-nitrophenoxy)phenyl]butanoate as a white solid (541 mg, 57%).

Methyl 3-[3,5-dibromo-4-(4-nitrophenoxy)phenyl) butanoate was dissolved in acetic acid (18 mL) and water (2 mL), and iron powder (310 mg, 5 eq.) was added. The reaction mixture was stirred at room temperature under nitrogen for 17 h. The solvents were evaporated under vacuum and the residue was partionated between water and ethyl acetate. The water phase was extracted with ethyl acetate (2×10 mL) and the combined organic phases were washed with brine and dried over sodium sulphate. After filtration and evaporation, the crude was purified by flash chromatography (dichloromethane/methanol 10:0 to 9:1). Evaporation gave the title compound (310 mg, 64%) as a white solid.

### Intermediate 3

### Ethyl [4-(4-aminophenoxy)-3,5-dibromophenoxylacetate

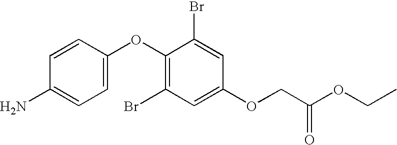

Sodium methoxide (2.2 g, 40 mmol) was added to a solution of 1,3-dibromo-5-fluoro-2-(4-nitrophenoxy)benzene (4 g, 10 mmol) in dimethylformamide (15 mL) at room temperature. The mixture was stirred at room temperature for 4 h. Water (20 mL) was added to the mixture and the product was extracted with ethyl acetate. The combined organic phases were washed consecutively with diluted hydrochloric acid and brine, dried over anhydrous magnesium sulphate and concentrated in vacuo. This crude mixture was used immediately without further purification.

Boron trifluoride-methyl sulfide complex (1 M, 12.8 mL, 12.8 mmol) was added dropwise to a stirred, chilled (dry ice-acetone bath) solution of crude 1,3-dibromo-5-methoxy-2-(4-nitrophenoxy)benzene (4.9 g, 12 mmol) in dichloromethane (150 mL). The mixture was allowed to warm up to room temperature and was stirred overnight. The reaction mixture was concentrated under vacuum, diluted with water, and extracted with ethyl acetate. The combined organic phases were washed with diluted hydrochloric acid, saturated sodium bicarbonate and brine, dried over anhydrous magnesium sulphate and concentrated in vacuo. The residue was purified by flash chromatography (heptane/ethyl acetate 20:1) to give 2.5 g (64.3%) of 3,5-dibromo-4-(4-nitrophenoxy)phenol as light yellow oil.

Ethyl bromoacetate (2.5 mL, 22 mmol) was added to a mixture of 3,5-dibromo-4-(4-nitrophenoxy)phenol (5.2 g, 13 mmol) and potassium carbonate (7.6 g, 54 mmol) in acetone (150 mL) at 0° C. After being stirred at ambient temperature for 4 h, the mixture was concentrated in vacuo. Ethyl acetate was added to the residue and the organic phase was washed with brine, dried over anhydrous magnesium sulphate and concentrated in vacuo to give the crude mixture of ethyl [3,5-dibromo-4-(4-nitrophenoxy)phenoxy]acetate which was used without further purification. To a solution of ethyl [3,5-dibromo-4-(4-nitrophenoxy)phenoxy]acetate (3.8 g, 8 mmol) in ethanol (150 mL), tin(II) chloride (9 g, 47 mmol) was added and the reaction mixture was stirred overnight at 80° C. After cooling to room temperature, the mixture was concentrated in vacuo and ethyl acetate and water were added to the residue. The organic phase was washed with sodium hydroxide (25% aqueous) and brine, dried over anhydrous potassium carbonate and concentrated in vacuo. The residue was purified by flash chromatography (heptane/ethyl acetate 4:1 to 2:1) to give 1.2 g (64.3%) of ethyl [4-(4-aminophenoxy)-3,5-dibromophenoxy]acetate.

### Intermediate 4

### Methyl 2-fluoro-3-13,5-dibromo-4-(4aminophenoxy)phenyl]propanoate

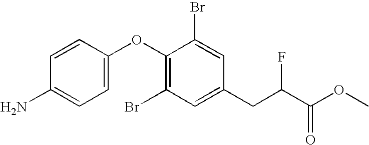

Sodium hydride (242 mg, 7.06 mmol, 70%) was dissolved in dry methanol (30 mL) with stirring. Methyl 2-hydroxy-3-(4-hydroxy-3,5-dibromophenyl) propanoate (2.5 g, 7.06 mmol) was added to the solution at room temperature and the solvent was evaporated off under reduced pressure to give the sodium phenolate as a white solid.

The phenolate and p-dinitrobenzene (1.19 g, 7.06 mmol) were dissolved in dimethyl sulphoxide (25 mL). The reaction mixture was heated to 90° C. for 15 h under a calcium chloride guard tube. The reaction mixture was poured into ice-water (150 mL) and extracted with ethyl acetate (3×100 mL). The combined organic phases were washed with sodium hydroxide (50 mL, aqueous 1M) and brine and dried over sodium sulphate, filtrated and evaporated. The crude was purified by chromatography on silica gel (ethyl acetate/petroleum 1:9 to 2:8) to afford 1.39 g of methyl 2-hydroxy-3-[3,5-dibromo-4-(4-nitrophenoxy)phenyl]propanoate (43% yield). A solution of methyl 2-hydroxy-3-[3,5-dibromo-4-(4-nitrophenoxy)phenyl]propanoate (1.39 g, 2.93 mmol) in dry dichloromethane (15 mL) was added dropwise to the solution of DAST (Et₂NSF₃) (0.5 g, 3.13 mmol) in dry dichloromethane (10 mL) at 0° C. under nitrogen atmosphere. The mixture was stirred for 15 min and allowed to come to room temperature and poured into a mixture of ice and water. The organic layer was separated and the water was extracted with dichloromethane (2×60 mL). The combined organic layers were washed with brine and dried over sodium sulphate, filtrated and evaporated. Methyl 2-fluoro-3-[3,5-dibromo-4-(4-nitrophenoxy)phenyl]propanoate (1.62 g) was pure enough to be used in the next step without further purification.

Pd/C (10%, 80 mg) was added to a solution of methyl 2-fluoro-3-[3,5-dibromo-4-(4-nitrophenoxy)phenyl]propanoate (1.62 g) in methanol (150 mL). The reaction mixture was stirred under H₂ at room temperature until the starting material disappeared. The mixture was filtered through celite and the solvent evaporated under vacuum. The residue was purified by chromatography on silica gel (ethyl acetate/heptane 2:8 to 3:7) to afford 1.15 g of methyl 2-fluoro-3-[3,5-dibromo-4-(4-aminophenoxy)phenyl]propanoate as a yellow solid (80% yield for two steps).

### Intermediate 5

### Methyl N-[4-(4-aminophenoxy)-3,5-dibromobenzoyl]glycinate

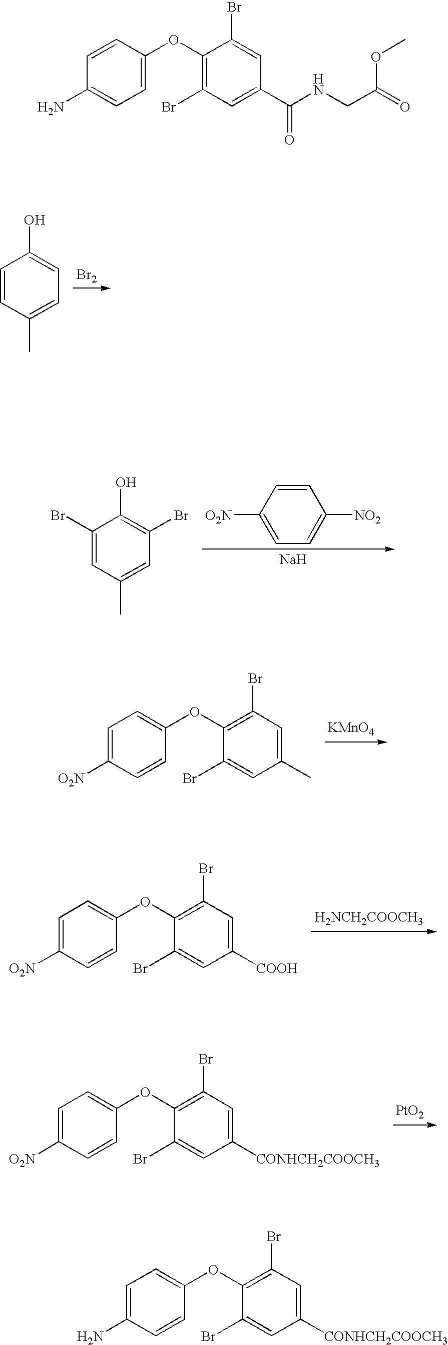

Bromine (5.75 ml, 54.6 mmol) in glacial acetic acid (80 mL) was added dropwise into a solution of p-cresol (5.9 g, 54.6 mmol) in acetic acid (12 mL) and water (33 mL) in a water-bath. The reaction solution was stirred for additional 0.5 h at room temperature and poured into water (200 mL). The precipitate was collected and purified by recrystallisation from ethyl acetate/petroleum ether. 2,6-Dibromo-4-methyl-phenol (13.2 g) was obtained in 91% yield.

Sodium hydride (0.46 g, 13.4 mmol) was pre-washed with hexane (to remove coal oil/kerosene) and was carefully dissolved in methanol (41 mL, anhydrous). 2,6-Dibromo-4-methyl-phenol (3.43 g, 12 mmol) was added to the basic solution. The solvent was evaporated to obtain a white solid which was mixed with dimethylsulfoxide (18.5 mL, anhydrous) and p-dinitrobenzene (1.90 g, 11.3 mmol), and heated at 90° C. for 16 h (water free conditions). The reaction mixture was poured into 500 mL of water/ice, and extracted with diethyl ether (3×500 mL). The combined organic phases were washed with aqueous sodium hydroxide (5%, 200 mL) and water (200 ml), dried and concentrated. The residue was purified by flash chromatography to give 1,3-dibromo-5-methyl-2-(4-nitro-phenoxy)-benzene (2.6 g) in 59% yield.

1,3-Dibromo-5-methyl-2-(4-nitro-phenoxy)-benzene (2.60 g, 6.7 mmol) was dissolved in pyridine (30 mL) and water (12 mL), and refluxed. Potassium permanganate(8.5 g, 53.8 mmol) was added in portions to the refluxing solution and the mixture was allowed to cool down and stirred for 6 h at room temperature. The reaction solution was diluted with ethyl acetate and filtered through celite. The residue obtained after evaporation of the solvent was diluted with hydrochloric acid (2 M) and extracted with ethyl acetate. The combined organic phases were saponified with sodium hydroxide (5%, aqueous). The alkaline aqueous solution was acidified with hydrochloric acid and extracted with acetate. The combined ethyl acetate phases were dried and concentrated to give 3,5-dibromo-4-(4-nitro-phenoxy)-benzoic acid (2.0 g) in 71% yield.

3,5-Dibromo-4-(4-nitro-phenoxy)-benzoic acid (1.6 g, 3.85 mmol), glycine methyl ester (hydrochloride salt, 1.54 g, 4.17 mmol), 3-ethyl-1-[3-(dimethylamino)propyl]carbodiimide hydrochloride (EDCI) (3.22 g, 5.77 mmol), and 1-hydroxybenzotriazole hydrate (HOBt) (2.27 g, 5.76 mmol) were dissolved in anhydrous dichloromethane (50 mL). After the addition of triethylamine (1.6 mL, 11.5 mmol), the reaction mixture was stirred overnight at room temperature. Direct purification by flash chromatography gave methyl N-[4-(4-nitrophenoxy)-3,5-dibromobenzoyl]glycinate (1.4 g) in a 75% yield.

Methyl N-[4-(4-nitrophenoxy)-3,5-dibromobenzoyl]glycinate (1.45 g, 3 mmol) and platinum oxide (72.5 mg, 0.3 mmol.) were suspended in ethyl acetate (120 mL), and hydrogenated under normal pressure for 30 h. The reaction solution was filtered and concentrated, purified by flash chromatography. Methyl N-[4-(4-aminophenoxy)-3,5-dibromobenzoyl]glycinate (1.0 g) was obtained in 75% yield.

### Intermediate 6

### Methyl 3-[4-(4-aminophenoxy)-3,5dichlorophenyl]propanoate

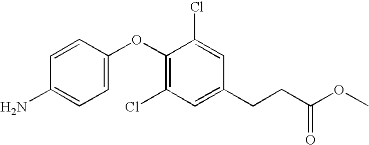

A solution of p-fluoro nitrobenzene (500 mg, 3.5 mmol), methyl 3-(4-hydroxy-3,5-dichlorophenyl) propanoate (500 mg, 2 mmol), copper powder (243 mg, 3.8 mmol) and potassium carbonate (630 mg, 4.5 mmol) in dimethylformamide (7 mL) was heated at 100° C. for 3 h. The cooled reaction mixture was diluted with ethyl acetate and hydrochloric acid (1 M) and extracted with ethyl acetate (3×10 mL). The combined organic phases were concentrated and filtered through a short flash chromatography column (heptane/ethyl acetate 10:0 to 5:5) to give methyl 3-[3,5-dichloro-4-(4-nitrophenoxy)phenyl]propanoate (600 mg, yield: 80%).

To a stirred solution of methyl 3-[3,5-dichloro4-(4-nitrophenoxy)phenyl]propanoate (600 mg, 1.6 mmol) in ethanol (50 mL) and water (3 mL), tin(II) chloride (1.83 g, 8 mmol) was added. The reaction mixture was stirred for 17 h at 90° C. Ethanol was removed under vacuum and the residue was diluted with ethyl acetate (50 mL) and a saturated solution of sodium carbonate (50 mL) and extracted with ethyl acetate (2×10 mL). The combined ethyl acetate layers were washed with brine, dried over sodium sulphate and concentrated. The residue was purified by flash chromatography (heptane/ethyl acetate 10:0 to 7:3) to afford the title compound (180 mg) in 35% yield.

### Intermediate 7

### [4-(4-Amino-phenoxy)-3,5-dichloro-benzoylaminol-acetic acid methyl ester

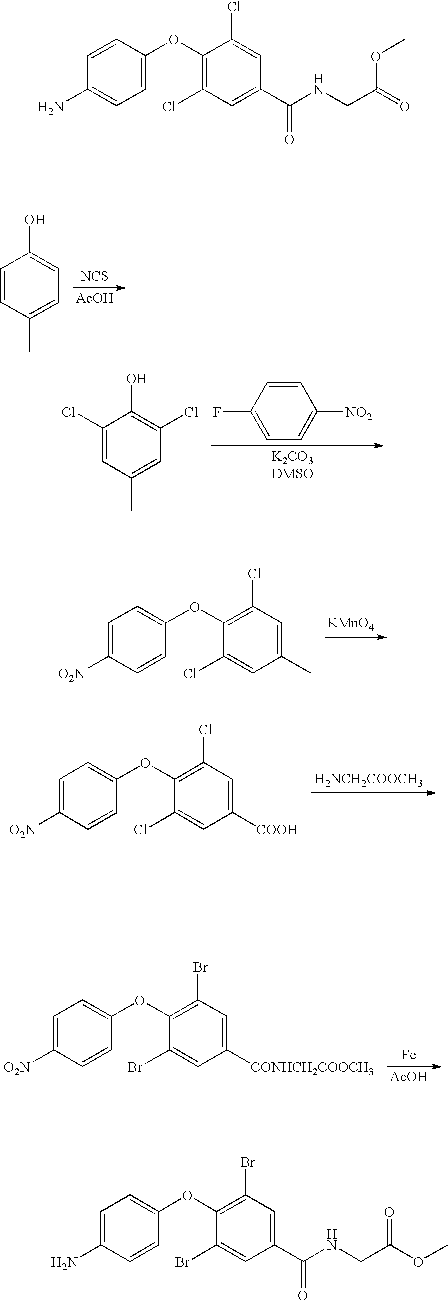

NCS (27.8 g, 208 mmol) was added to the solution of p-cresol (7.5 g, 69 mmol) in acetic acid (100 ml). The reaction mixture was heated to 70° C. and stirred for 48 h. The mixture was poured into water and the yellow precipitate was collected. The crude was purified by chromatography on silica gel with ethyl acetate and petroleum ether (1:100) to afford 6.8 g of 2,6-dichloro4-methyl-phenol (40% purity).

4-Fluro-nitrobenzene (4.65 g, 33 mmol), copper powder (2.3 g 35.6 mmol) and K₂CO₃ (6.3 g, 45.4 mmol) were added to a solution of 2,6-dichloro-4-methyl-phenol (40% pure) (6.6 g, 37.3 mmol) in DMSO (200 mL, dried with CaCl₂). The reaction mixture was heated to 120° C. and stirred overnight. After cooling to room temperature the reaction mixture was filtered through celite. The filtrate was partitioned between EtOAc and water and the water phase was extracted twice with EtOAc. The combined organic phases were washed with 1M NaOH, brine, dried over anhydrous Na₂SO₄ and filtered. The solvent was evaporated under reduced pressure to give a residue which was purified by chromatography on silica gel with ethyl acetate and petroleum ether (1:100-1:40) to afford 5.7 g 1,3-dichloro-5-methyl-2-(4-nitro-phenoxy)-benzene as a white solid.

A solution of 1,3-dichloro-5-methyl-2-(4-nitro-phenoxy)-benzene (5.7 g, 19 mmol) in pyridine (120 mL) and water (50 mL) was heated to reflux. KMnO₄ (28 g, 177 mmol) was added portionwise (8 h), and then the reaction mixture was refluxed overnight. The reaction solution was diluted with EtOAc and filtered through celite. The solution was concentrated, diluted with 2M HCl and extracted with EtOAc. The combined organic phase was washed with brine, dried over Na₂SO₄, and concentrated. The residue was purified by chromatography on silica gel with ethyl acetate and petroleum ether (1:20-1:5) to afford 3.8 g of 3,5-dichloro-4-(4-nitro-phenoxy)-benzoic acid as a white solid.

3,5-Dichloro-4-(4-nitro-phenoxy)-benzoic acid (3.8 g, 11.6 mmol), glycine methyl ester hydrochloride (2.07 g, 17.4 mmol), EDCI (3.16 g, 17.4 mmol), and HOBt (2.23 g, 17.4 mmol) were dissolved in 100 ml CH₂Cl₂. After the addition of 4.8 mL of Et₃N, the reaction mixture was stirred overnight at room temperature. 300 mL CH₂Cl₂ was added and then the reaction mixture was washed with 1M HCl, brine, dried over Na₂SO₄ and filtrated. The solvent was evaporated to give a residue which was purified by chromatography on silica gel with ethyl acetate and petroleum ether (1:3) to afford 3.5 g [3,5-dichloro-4-(4-nitro-phenoxy)-benzoylamino]-acetic acid methyl ester as a white solid.

TLC: petroleum ether/ethyl acetate/methanol=10:2:1 Rf=0.17

Fe powder (2.46 g, 44 mmol ) was added to the solution of [3,5-dichloro-4-(4-nitro-phenoxy)-benzoylamino]-acetic acid methyl ester (3.5 g, 8.8 mmol) in acetic acid (90 mL) and water (9 mL), the reaction mixture was stirred overnight. The reaction solution was filtered through celite and partitioned between EtOAc and H₂O. The organic phase was washed with water, saturated NaHCO₃ solution, saturated NaCl solution, dried over Na₂SO₄ and filtrated. The solvent was concentrated to give a residue which was purified by chromatography on silica gel with ethyl acetate and petroleum ether (1:2) to afford 2.9 g 4-(4-amino-phenoxy)-3,5-dichloro-benzoylamino)-acetic acid methyl ester in 11.3% yield.

TLC:petroleum ether/ethyl acetate=2:1 Rf=0.12

### Intermediate 8

### 3-[4-(4Amino-phenoxy)-3,5-dichlorophenyl]-2-fluoro-propionic acid methyl ester

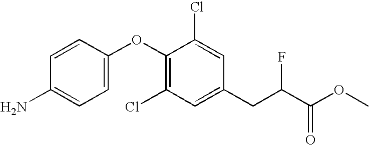

A solution of 2-hydroxy-3-(4-hydroxy-phenyl)-propionic acid methyl ester (17 g, 86.7 mmol) and NCS (23 g, 173.4 mmol) in 300 mL of AcOH was stirred for 2 h at 60° C. Water was added and the reaction mixture was extracted with EtOAc. The organic phase was washed with NaHCO₃, water, dried over Na₂SO₄, filtrated and evaporated. Purification of the product by column chromatography (2:8-3:7 EtOAc/petroleum ether) gave 7.5 g crude product. Sodium hydride (0.97 g, 28.4 mmol, 70%) was dissolved in dry methanol (100 mL) with stirring. 3-(3,5-dichloro-4-hydroxy-phenyl)-2-hydroxy-propionic acid methyl ester (7.5 g, 28.4 mmol) was added to the solution at room temperature and the solvent was evaporated off under reduced pressure to give the sodium salt as a white solid. p-Fluoro-nitrobenzene (3.2 g, 28.4 mmol) dissolved in 150 mL dimethyl sulphoxide was added. The reaction mixture was heated to 90° C. for 15 h. The reaction mixture was poured into ice-water (150 mL) and extracted with EtOAc (3*100 mL). The combined organic phases were washed with aqueous 1M sodium hydroxide (50 mL) and brine, dried over Na₂SO₄, filtrated and evaporated. The crude product was purified.

A solution of 3-[3,5-dichloro-4-(4-nitro-phenoxy)-phenyl]-2-hydroxy-propionic acid methyl ester (1.2 g, 3.1 mmol) in dry dichloromethane (50 mL) was added dropwise slowly to the solution of DAST (0.45 mL, 3.3 mmol) in dry dichloromethane (10 mL) at 0° C. under nitrogen atmosphere. The mixture was stirred for 15 min and allowed to come to room temperature and poured into a mixture of ice and water. The organic layer was separated and the water was extracted twice with dichloromethane. The combined organic layers were washed with brine and dried over Na₂SO₄. The 3-[3,5-dichloro-4-(4-nitro-phenoxy)-phenyl]-2-fluoro-propionic acid methyl ester (1 g) was pure enough to be used in the next step without further purification.

PtO₂ (0.3 g) was added to a solution of 3-[3,5-dichloro-4-(4-nitro-phenoxy)-phenyl]-2-fluoro-propionic acid methyl ester (1 g) in methanol (50 mL), and then the reaction mixture was stirred under H₂ at room temperature until the starting material disappeared. The mixture was filtered through celite and the solvent was evaporated. The crude product was purified by chromatography on silica gel with ethyl acetate and petroleum (2:8 -3:7) to afford 1 g of 3-[4-(4-amino-phenoxy)-3,5-dichloro-phenyl]-2-fluoro-propionic acid methyl ester

TLC: AcOEt:P.E.=1:1 Rf=0.3

### General Procedure A for the Preparation of Intermediates 9-15

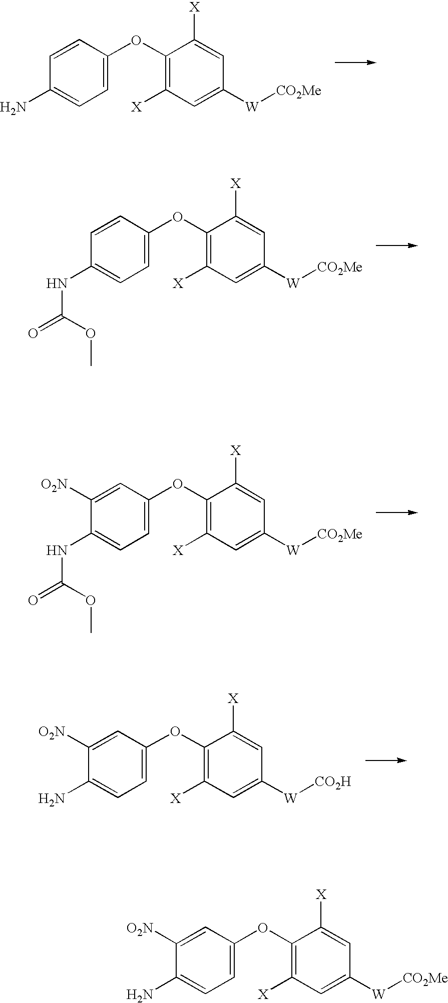

To a dichloromethane solution of the appropriate aniline (intermediates 1-8) (e.g. methyl 3-3,5-dibromo-4-[4-aminophenoxyjphenyl)propanoate) and pyridine) (2 eq.) was added dropwise methyl chloroformate (1.1 eq.). After stirring at room temperature for 1 h the reaction mixture was poured into ice-water. The mixture was extracted with dichloromethane and the combined organic phases were washed with brine and dried over sodium sulphate. The crude mixture was purified by flash chromatography (petroleum ether/ethyl acetate). The obtained residue contained the wanted carbamate (e.g. methyl 3-(3,5-dibromo4-[(4-methoxycarbonylamino)phenoxy]phenyl)propanoate). To a solution of the appropriate carbamate (crude mixture) (e.g. methyl 3-(3,5-dibromo4-[(4-methoxycarbonylamino)phenoxy]phenyl)propanoate) in acetic acid (50 mL/mmol) was added fuming nitric acid (0.4 mL/mmol). The solution was stirred at room temperature for 1.5 h. The reaction was quenched by careful addition of sodium bicarbonate (saturated aqueous solution). The mixture was extracted with ethyl acetate and the combined organic layers were washed with brine and dried over sodium sulphate. The solvent was evaporated under vacuum to give the wanted nitro derivative (e.g. methyl 3-(3,5-dibromo-4-[(4-methoxycarbonylamino)-3-nitrophenoxy]phenyl)propanoate) which could be used without further purification.

To a stirred solution of the appropriate nitro derivative (e.g. methyl 3-(3,5-dibromo-4-[(4-methoxycarbonylamino)-3-nitrophenoxy]phenyl)propanoate) in methanol (30 mL/mmol), aqueous sodium hydroxide (15%) was added (10 mL/mmol). The mixture was refluxed for 3 h. Methanol was removed under reduced pressure and water was added to the residue. The mixture was extracted with ethyl acetate. The combined organic phases were washed with brine and dried over sodium sulphate. The crude product containing the appropriate acid was used directly in the next step (e.g. 3-[4-(4-amino-3-nitrophenoxy)-3,5-dibromo-phenyl]propanoic acid).

To a methanol (30 mL/mmol) solution of the appropriate acid (e.g. 3-[4-(4-amino-3-nitrophenoxy)-3,5-dibromo-phenyl]propanoic acid), hydrochloric acid (37%) was added (0.4 mL/mmol). The reaction mixture was heated to reflux for 2 h. After evaporation of the solvent, the residue was dissolved in ethyl acetate. The organic layer was washed with sodium bicarbonate (aqueous solution) and brine and dried over sodium sulphate. The crude product was purified by flash chromatography (petroleum ether/ethyl acetate) to afford the appropriate ester (e.g. methyl 3-[4-(4-amino-3-nitrophenoxy)-3,5-dibromo-phenyl]propanoate).

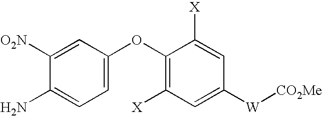

| | | | Yield | MW | |
|---|---|---|---|---|---|
| Intermediate | X | W | (%) | (calc) | M (found) |
| 9 | Br | (CH₂)₂ | 57 | 474.1 | 474.9 |
| 10 | Br | CH₂—CHF | 46 | 492.1 | 492.9 |
| 11 | Br | CONH—CH₂ | 56 | 503.1 | 503.6 |
| 12 | Br | NHCO—CH₂ | 34 | 503.1 | 503.6 |
| 13 | Br | O—CH₂ | 29 | 476.07 | 491.4 |
| | | | | | (M + NH₄) |
| 14 | Cl | CONH—CH₂ | 13 | 414.20 | 414.0 |
| 15 | Cl | CH₂—CHF | 36 | 403.19 | 490.2 |
| | | | | | (M + NH4) |

### Intermediate 16

### N-[4-(4-Amino-phenoxy)-3,5-dibromo-phenyl]-malonamic acid methyl ester

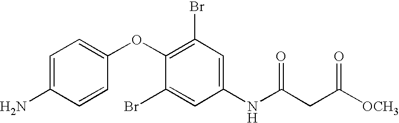

To a solution of 2,6-dibromo-4-nitrophenol (14.10 g, 47.49 mmol) in ethanol(200 mL), Tin (II) chloride dehydrate(53.6 g, 237.5 mmol) was added. After stirring at 60° C. overnight the reaction mixture was cooled to room temperature and ethanol was evaporated. The reaction mixture was poured into aqueous 25% sodium hydroxide (150 mL) and then filtered through celite. The filtrate was extracted with EtOAc (3*200 mL). The combined organic phases were washed with brine (200 mL), dried over Na₂SO₄ filtrated and evaporated to afford 4-amino-2,6-dibromo-phenol (10.77 g) in 80% of yield.

Sodium hydride (1.22 g, 30.49 mmol, 60%) was dissolved in dry methanol (50 mL) with stirring. The 4-amino-2,6-dibromo-phenol (8.14 g, 30.49 mmol) was added to the solution at room temperature and the solvent was evaporated off under reduced pressure to give the phenoxide as a black solid.

The phenoxide and the 1,4-dinitrobenzene (4.43 g, 26.22 mmol) were dissolved in 100 mL dimethyl sulphoxide. The reaction mixture was heated to 90° C. for 20 h under a calcium chloride guard tube. The reaction mixture was poured into ice-water (400 mL) and extracted with EtOAc (4*300 mL). The combined organic phases were washed with brine, dried over Na₂SO₄. The crude product was purified by chromatography on silica gel with ethyl acetate and heptane (2:8) to afford 5.78 g of 3,5-dibromo-4-(4-nitro-phenoxy)-phenylamine in 49% yield.

To a solution of 3,5-dibromo4-(4-nitro-phenoxy)-phenylamine (5.78 g, 14.9 mmol) in dry dichloromethane (100 mL), pyridine (2.4 mL, 29.8 mmol) and methyl 3-chloro-3-oxopropionate (1.6 mL, 14.9 mmol) was added. After 0.5 h stirring at room temperature, dichloromethane was evaporated, EtOAc was added and the mixture was washed with water followed by saturated brine. The organic phase was dried over Na₂SO₄, filtrated and then concentrated to a yellow solid. The crude product was purified by chromatography on silica gel with ethyl acetate and heptane (2:8-3:7) to afford 3.48 g of compound N-[4-(4-Amino-phenoxy)-3,5-dibromo-phenyl]-malonamic acid methyl ester in 48% of yield.

### General Procedure A1 for the PreDaration of Examples 1 and 3

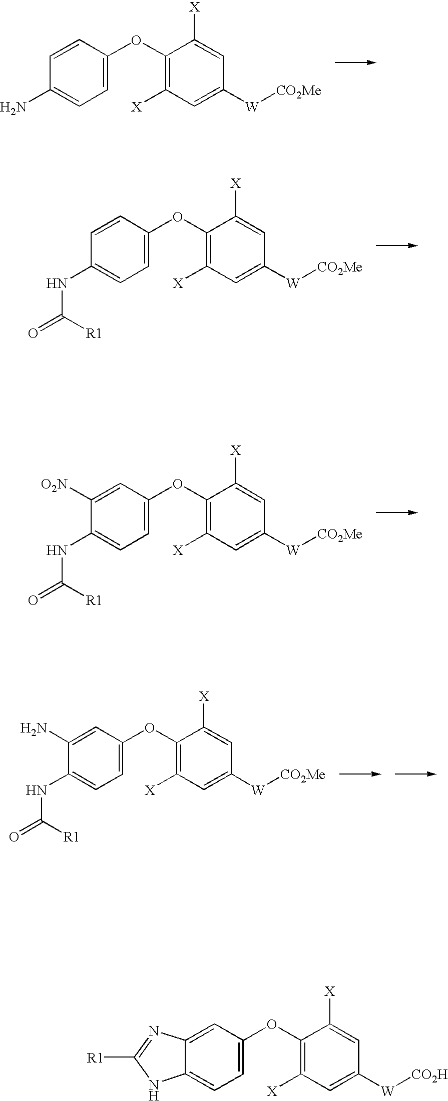

To an acetic acid solution(8 mL/mmol) of the appropriate aniline (e.g. methyl 3-(3,5-dibromo-4-[4-aminophenoxy]phenyl) propanoate) at ambient temperature, was added the appropriate anhydride (e.g. acetic acid anhydride) (1.1 eq.). The reaction mixture was followed by TLC and after 45 minutes the appropriate amide had formed (e.g. 3-[4-(4-acetylamino-3-nitro-phenoxy)-3,5-dibromo-phenyl]-propionic acid methyl ester). The reaction mixture was cooled at 0° C. and sulphuric acid (4 mL/mmol) was added. After additional 10 minutes cooling, fuming nitric acid (1.1 eq.) was added. The solution was stirred at 0° C. for 1 h and then at room temperature for 17 h. The reaction was quenched by addition of ice. The mixture was extracted with ethyl acetate and the combined organic layers were washed with brine and dried over sodium sulphate. The solvent was evaporated under vacuum to give the wanted nitro derivative (e.g. 3-[4-(4-Acetylamino-3-nitro-phenoxy)-3,5-dibromo-phenyl]-propionic acid methyl ester) which could be used without further purification.

Platinum oxide (0.4 eq.) was added to an ethyl acetate solution (27 ml/mmol) of the appropriate nitro derivative (e.g. 3-[4-(4-acetylamino-3-nitro-phenoxy)-3,5-dibromo-phenyl]-propionic acid methyl ester) and the mixture was stirred under a 1.2 bar pressure of hydrogen for 17 h at 20° C. The reaction mixture was filtered through a celite pad which was rinsed with ethyl acetate. Evaporation of the solvent gave the wanted aniline (e.g. 3-[4-(4-acetylamino-3-amino-phenoxy)-3,5-dibromo-phenyl]-propionic acid methyl ester) which could be used without further purification. A solution of the appropriate aniline derivative (crude mixture) (e.g. −[4-(4-acetylamino-3-amino-phenoxy)-3,5-dibromo-phenyl]-propionic acid methyl ester) in acetic acid (50 mL/mmol) was stirred for 17 h at 80° C. After evaporation of the acetic acid, the residue was dissolved in tetrahydrofuran and lithium hydroxide (1 M) was added. The mixture was stirred for 17 h at 20° C. After acidification with hydrochloric acid (2 M), the product was extracted into ethyl acetate. The combined organic layers were washed with brine and dried over sodium sulphate. After filtration, the residue was purified by semi-preparative-HPLC (Zorbax CombiHT (SB-C8 50×21.2 mm, 5μ). Mobile Phase: Solvent A. Water with 0.5% formic acid; Solvent B: acetonitrile. Gradient: 2 min 80% of A then over 8 min to 5% of A) to give the expected acid (e.g. 3-[3,5-dibromo-4-(2-methyl-1H-benzoimidazol-5-yloxy)-phenyl]-propionic acid (example 1)).

### Example 2

3-{3,5-Dibromo-4-[2-(3-methoxy-phenyl)-1H-benzoimidazol-5-yloxy]-phenyl}-propionic acid To DMA solution (43 mL/mmol) of the aniline derivative 3-[4-(4-Acetylamino-3-amino-phenoxy)-3,5-dibromo-phenyl]-propionic acid methyl ester generated according to procedure A1, was added sodium hydrogen carbonate (1.5 eq.) and 3-methoxy-benzaldehyde (1.1 eq.). The reaction was heated to 160° C. for 6 h in a bomb. EtOAc was added and the organic phase was washed with H₂O, brine, dried over sodium sulphate and filtrated. The solvent was evaporated and the residue was purified by preparative HPLC (Zorbax CombiHT(SB-C8 50×21.2 mm, 5μ). Mobile Phase: Solvent A. Water with 0.5% formic acid; Solvent B: acetonitrile. Gradient: 2 min 80% of A then over 8 min to 5% of A). The combined fractions were evaporated, the residue was dissolved in tetrahydrofuran, and lithium hydroxide (1M) was added. The mixture was stirred for 17 h at 20° C. After acidification with hydrochloric acid (2 M), the product was extracted into ethyl acetate. The combined organic layers were washed with brine and dried over sodium sulphate. After filtration, the residue was purified by semi-preparative-HPLC (Zorbax CombiHT (SB-C8 50×21.2 mm, 5μ). Mobile Phase: Solvent A. Water with 0.5% formic acid; Solvent B: acetonitrile. Gradient: 2 min 80% of A then over 8 min to 5% of A) to give the expected 3-{3,5-Dibromo-4-[2-(3-methoxy-phenyl)-1H-benzoimidazol-5-yloxy]-phenyl}-propionic acid) as a solid.

### General Procedure A2 for the Preparation of Examples 4-19, 22-23

To a dichloromethane solution of the appropriate aniline (e.g. methyl 4-[4-(4-aminophenoxy)-3,5-dibromophenyl]butanoate) was added pyridine (10 mL/mmol) and the appropriate acid chloride (e.g. 4-methyl-benzoyl chloride) (1.2 eq.). The mixture was stirred at room temperature for 17 h. After acidification with hydrochloric acid (2 M, aqueous solution), the product was extracted with chloroform using a phase separator. The solvents were evaporated and the last remains of pyridine were coevaporated with toluene. The obtained residue contained the wanted amide (e.g. 4-{3,5-Dibromo-4-[4-(4-methyl-benzoylamino)-3-nitro-phenoxy]-phenyl) -butyric acid methyl ester) and was used without further purification.

The appropriate nitro derivative (e.g. methyl 4-(3,5-dibromo-4-(4-[(4-methylbenzoyl)amino]-3-nitrophenoxy}phenyl)butanoate) (1 eq.) was dissolved in acetic acid-water (9:1, 70 mL/mmol) and iron powder (5 eq.) was added. The reaction mixture was heated to reflux overnight. The acetic acid was evaporated and partionated between ethyl acetate and hydrochloric acid (1 M). The organic phase was washed with water and brine and then dried over sodium sulphate. After filtration and evaporation of the solvent, the residue was dissolved in tetrahydrofuran (30 mL/mmol) and lithium hydroxide (1 M, 10 mL/mmol) was added. The reaction mixture was stirred at room temperature for 17 h. After acidification with HCl (2 M), the product was extracted with ethyl acetate and dried with sodium sulphate, filtrated and evaporated. The obtained residue was purified by semi-preparative-HPLC (Zorbax CombiHT (SB-C8 50×21.2 mm, 5μ). Mobile Phase: Solvent A. Water with 0.5% formic acid; Solvent B: acetonitrile. Gradient: 2 min 80% of A then over 8 min to 5% of A) to give the expected acid (e.g. 4-(3,5-dibromo-4-{[2-(4-methylphenyl)-1H-benzimidazol-5-yl]oxy)phenyl)butanoic acid).

### General Procedure B1 for the Preparation of Examples 20-21

To a dichloromethane (50 mL/mmol) solution of the appropriate nitro-aniline (intermediate 9-15) (e.g. methyl 3-[4-4-amino-3-nitrophenoxy)-3,5-dibromo-phenyl]propanoate) was added pyridine (2.2 eq.) and the appropriate acid chloride (e.g. 3-methylthiopropionyl-chloride) (2.2 eq.). The mixture was stirred at room temperature for 17 h. After acidification with hydrochloric acid (2 M, aqueous solution), the product was extracted with dichloromethane using a phase separator. The solvents were evaporated and the last remains of pyridine were coevaporated with toluene. The obtained residue contained the wanted amide (e.g. methyl 3-{4-[4-(3-methylthio-propionyl)amino-3-nitrophenoxy]-3,5-dibromo-phenyl}-propanoate) and was used without further purification. The residue was dissolved in ethanol (50 mL/mmol) and hydrochloric acid (conc., 50 μL/mmol) and tin(II) chloride monohydrate (5 eq.) were added. The reaction mixture was stirred for 17 h at 80° C. under nitrogen atmosphere. Ethanol was removed under vacuum and the residue was diluted with ethyl acetate and saturated solution of sodium carbonate and extracted with ethyl acetate. The combined ethyl acetate layers were washed with brine, dried over sodium sulphate and concentrated. The residue was dissolved in tetrahydrofuran (20 mL/mmol) and treated with lithium hydroxide (1M, 10 mL/mmol). The mixture was stirred at room temperature for 7 h. After acidification with HCl (2M), the product was extracted with ethyl acetate and dried with sodium sulphate. The obtained residue was purified by semi-preparative-HPLC (Zorbax CombiHT (SB-C8 50×21.2 mm, 5μ). Mobile Phase: Solvent A. Water with 0.5% formic acid; Solvent B: acetonitrile. Gradient: 2 min 80% of A then over 8 min to 5% of A) to give the expected acid (e.g. 3-(3,5-dibromo-4-{[2-(2-(methylthio)-ethyl)-1H-benzimidazol-5-yl]oxy}phenyl)propanoic acid.

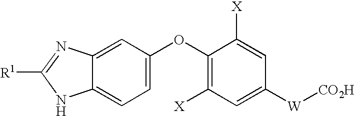

| | | | | Yield | MW | M + 1 |
|---|---|---|---|---|---|---|
| Example | R¹ | X | W | (%) | (calc) | (found) |
| 1 | Me | Br | (CH₂)₂ | 7 | 454.1 | 455.0 |
| 2 | m-OMe-Ph | Br | (CH₂)₂ | 4 | 546.2 | 547.2 |
| 3 | i-Bu | Br | O—CH₂ | 24 | 498.2 | 499.1 |
| 4 | p-Me-Ph | Br | (CH₂)₃ | 14 | 544.3 | 545.3 |
| 5 | p-F-Ph | Br | (CH₂)₃ | 10 | 548.2 | 549.5 |
| 6 | p-Cl-Ph | Br | (CH₂)₃ | 50 | 564.7 | 565.4 |
| 7 | m-Cl-Ph | Br | (CH₂)₃ | 50 | 564.7 | 565.4 |
| 8 | m,p-diMe-Ph | Br | (CH₂)₂ | 10 | 544.3 | 545.3 |
| 9 | m-F, p-Me-Ph | Br | (CH₂)₂ | 8 | 548.2 | 549.5 |
| 10 | i-Bu | Br | CH₂—CHF | 60 | 514.2 | 515.3 |
| 11 | m-Me-Ph | Br | (CH₂)₂ | 3 | 530.2 | 531.2 |
| 12 | m-Me, p-F-Ph | Br | CH₂—CHF | 4 | 566.2 | 565.4 |
| | | | | | | (M − 1) |
| 13 | p-Me-Ph | Br | CH₂—CHF | 4 | 548.2 | 549.5 |
| 14 | m-Me, p-F-Ph | Br | O—CH₂ | 5 | 550.2 | 551.6 |
| 15 | t-Bu-methyl | Br | O—CH₂ | 43 | 512.2 | 513.5 |
| 16 | Cyclopropyl- | Br | O—CH₂ | 41 | 524.2 | 525.5 |
| | methyl | | | | | |
| 17 | n-Pr | Br | O—CH₂ | 46 | 484.2 | 485.3 |
| 18 | m-Me-Ph | Cl | (CH₂)₂ | 20 | 441.3 | 441.5 |
| | | | | | | (M) |
| 19 | 2-Methylthio- | Br | CONH— | 9 | 543.2 | 544 |
| | ethyl | | CH₂ | | | |
| 20 | 2-Methylthio- | Br | (CH₂)₂ | 8 | 514.2 | 514.2 |
| | ethyl | | | | | (M) |
| 21 | 2-Methylthio- | Br | CH₂—CHF | 13 | 532.2 | 533 |
| | ethyl | | | | | |
| 22 | cyclopentyl- | Br | O—CH₂ | 43 | 524.22 | 525.5 |
| | methyl | | | | | |
| 23 | m-F, p-Me-Ph | Br | CH₂—CHF | 26 | 566.21 | 567.62 |

### Examples 24-27

### 3-(3,5-dibromo-4-{[2-isopropylcarbamoyl-1H-benzimidazol-5-yl[oxy}phenyl)-2-fluoropropanoic acid

### 3-(3,5-dibromo-4-{[2-ethylcarbamoyl-1H-benzimidazol-5-yl]oxy}phenyl)-2-fluoropropanoic acid

### 3-(3,5-dibromo-4-{[2-diisopropylcarbamoyl-1H-benzimidazol-5-yl]oxy}phenyl)-2-fluoropropanoic acid

### 5-[2,6-Dibromo-4(2-fluoro-2-isopropylcarbamoyl-ethyl)-phenoxy]-1H-benzoimidazole-2-carboxylic acid

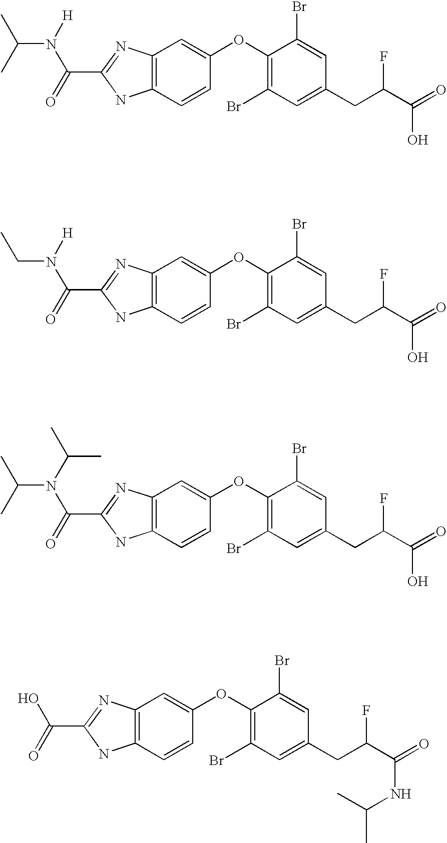

Methyl 3-[4-(4-amino-3-nitrophenoxy)-3,5-dibromo-phenyl]-2-fluoro-propanoate (intermediate 10) (22 mg, 0.045 mmol) and platinum oxide (2.3 mg, 0.010 mmol) were suspended in ethyl acetate (2 mL), and hydrogenated under pressure (5 psi) for 15 h. The reaction mixture was filtered through a celite pad and concentrated, purified by flash chromatography (methanol/dichloromethane 0:10 to 5:5) to give 19 mg (92% yield) of methyl 3-[4-(4,3-diarnino-phenoxy)-3,5-dibromo-phenyl]-2-fluoro-propanoate.

Methyl 3-[4-(4,3-diamino-phenoxy)-3,5-dibromo-phenyl]-2-fluoro-propanoate was dissolved in acetic acid (0.1 mL) and cooled to 0° C., methyl trichloroacetimidate (Cl₃CC(NH)OMe) (6 μL, 0.045 mmol) was added. The reaction was stirred for 1 h at room temperature. Water was added and the product was extracted with dichloromethane using a phase separator to give methyl 3-(3,5-dibromo-4-([2-trichloromethyl-1H-benzimidazol-5-yl]oxy}phenyl)-2-fluoropropanoate which was used in the next step without further purification.

The crude methyl 3-(3,5-dibromo4-{[2-trichloromethyl-1H-benzimidazol-5-yl]oxy}phenyl)-2-fluoropropanoate was dissolved in 1 mL of a stock solution (100 μL isopropylamine, 162 μL triethylamine, 24 mL ethanol) and stirred at room temperature over night. Potassium hydroxide (2 mL, 2 M) was added and the stirring continued for 1 h. Hydrochloric acid (1 M, 10 mL) was added and the mixture was extracted into ethyl acetate (3×30 mL). The combined organic phases were dried, filtered and concentrated. The obtained residue was purified by semi-preparative-HPLC (Zorbax CombiHT (SB-C8 50×21.2 mm, 5μ). Mobile Phase: Solvent A. Water with 0.5% formic acid; Solvent B: acetonitrile. Gradient: 2 min 80% of A then over 8 min to 5% of A) to give 1 mg (5% yield over 3 steps) of 3-(3,5-dibromo-4-{[2-isopropylcarbamoyl-1H-benzimidazol-5-yl]oxy}phenyl)-2-fluoropropanoic acid and also unexpected 1 mg of 3-(3,5-dibromo-4-{[2-ethylcarbamoyl-1H-benzimidazol-5-yl]oxy}phenyl)-2-fluoropropanoic acid, 0.8 mg of 3-(3,5-dibromo-4-{[2-diisopropylcarbamoyl-1H-benzimidazol-5-yl]oxy}phenyl)-2-fluoropropanoic acid and 0.7 mg of 5-[2,6-Dibromo-4-(2-fluoro-2-isopropylcarbamoyi-ethyl)-phenoxy-1H-benzoimidazole-2-carboxylic acid.

### Example 28

### 3-{3,5-Dibromo-4-[2-(methylsulfonylamino-methyl)-1H-benzoimidazol-5-yloxy]-phenyl{-propionic acid

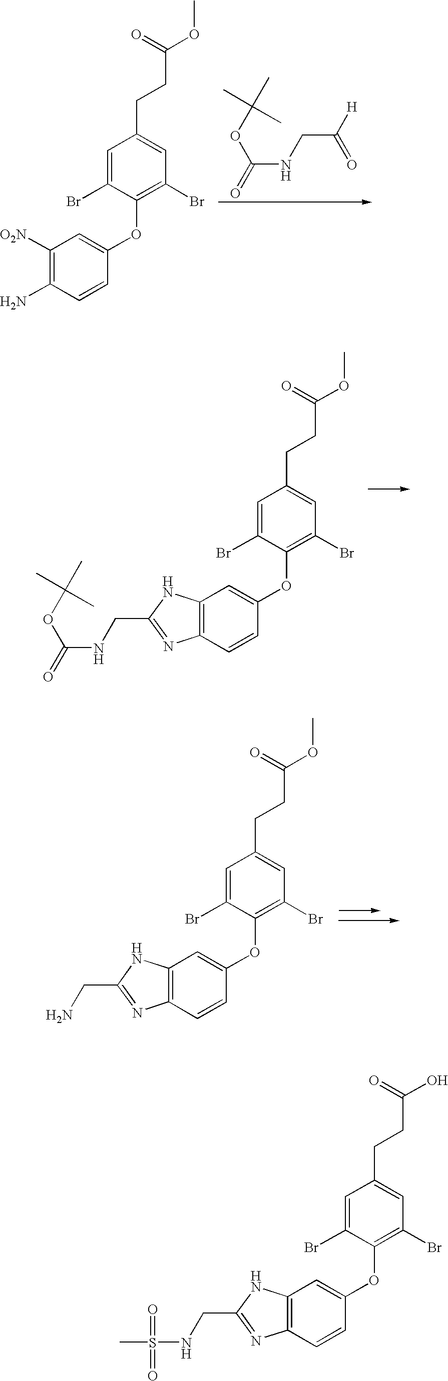

A mixture of intermediate 9 (33 mg, 0.07 mmol), t-BuCO₂NCH₂CHO (11 mg, 0.07mmol) and sodium hydrosulphite (Na₂S₂O₄) (36 mg, 0.21 mmol) in ethanol (1 mL), was heated at 80° C. for 16 h. After cooling, the mixture was diluted with a solution of sodium carbonate (saturated aqueous solution) and extracted with ethyl acetate. The combined organic layers were dried over magnesium sulphate, and concentrated in vacuo. The residual oil was dissolved in dichloromethane (4 mL) and trifluoroacetic acid (2 mL) was added. The mixture was stirred for 12 h at room temperature. The reaction was concentrated in vacuo and purified by semi-preparative-HPLC (Zorbax CombiHT (SB-C8 50×21.2 mm, 5μ). Mobile Phase: Solvent A. Water with 0.5% formic acid; Solvent B: acetonitrile. Gradient: 2 min 80% of A then over 8 min to 5% of A).

The obtained product was dissolved in dichloromethane (1 mL). Pyridine (14 μL, 0.07 mmol) and mesylchloride (5 μL, 0.07 mmol) were added. The reaction was stirred for 12 h at room temperature. Water and dichloromethane were added and the organic phase was collected using a phase separator. The solvent was evaporated under vacuum and the resulting oil residue was redissolved in dioxane (0.5 mL) and potassium hydroxide (2M, 1.5 mL) was added. The reaction mixture was stirred for 1 h, concentrated and the residue purified by semi-preparative-HPLC (Zorbax CombiHT (SB-C8 50×21.2 mm, 5μ). Mobile Phase: Solvent A. Water with 0.5% formic acid; Solvent B: acetonitrile. Gradient: 2 min 80% of A then over 8 min to 5% of A) to yield the title compound (2.16 mg) (6% yield over 4 steps).

### General Procedure B2 for the Preparation of Examples 29-42

To a dichloromethane (50 mL/mmol) solution of the appropriate nitro-aniline (intermediate 9-15) (e.g. 3-[4-(4-Amino-3-nitro-phenoxy)-3,5-dibromo-phenyl]-2-fluoro-propionic acid methyl ester) was added pyridine (2.2-4 eq.) and the appropriate acid chloride (e.g. 2,2,3,3-Tetrafluoro-propionyl chloride) (2.2-4 eq.). The mixture was stirred at room temperature for 17-72 h. After acidification with hydrochloric acid (2 M, aqueous solution), the DCM phase was separated using an isolute phase separator. The solvent was removed under a flow of nitrogen. The obtained residue contained the wanted amide (e.g. 3-{3,5-dibromo-4-[3-nitro-4-(2,2,3,3-tetrafluoro-propionylamino)-phenoxy]-phenyl}-2-fluoro-propionic acid methyl ester) and was used without further purification. The residue was dissolved in AcOH/H₂O and 29 mg of Fe (approx. 5 eq.) was added. The mixtures were stirred under reflux for 17 h. The solvent was removed in vacuo. The residue was dissolved in DMSO and filtered before purification by semi-preparative-HPLC (Zorbax CombiHT (SB-C8 50×21.2 mm, 5μ). Mobile Phase: Solvent A. Water with 0.5% formic acid; Solvent B: acetonitrile. Gradient: 20-100% ACN; 40 min 50 mI/min to give the expected acid (e.g. 3-{3,5-Dibromo-4-[2-(1,1,2,2-tetrafluoro-ethyl)-1H-benzoimidazol-5-yloxy]-phenyl}-2-fluoro-propionic acid).

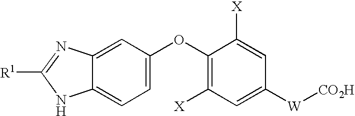

| Ex- | | | | | | |
|---|---|---|---|---|---|---|
| am- | | | | Yield | MW | M + 1 |
| ple | R¹ | X | W | (%) | (calc) | (found) |
| 29 | CHF₂CF₂ | Br | CH₂—CHF | 25 | 558.1 | 559.1 |
| 30 | CHF₂CF₂ | Br | CONH—CH₂ | 11 | 569.1 | 570.2 |
| 31 | CHF₂CF₂ | Br | O—CH₂ | 11 | 542.1 | 543.1 |
| 32 | 2-(2,5-dimethyl- | Br | CH₂—CHF | 45 | 553.2 | 554.3 |
| | oxa | | | | | |
| | zol-4-yl | | | | | |
| 33 | 2-(2,5-dimethyl- | Br | CONH—CH₂ | 21 | 564.2 | 565.4 |
| | oxa | | | | | |
| | zol-4-yl | | | | | |
| 34 | 2-(2,5-dimethyl- | Br | O—CH₂ | 4 | 537.2 | 538.4 |
| | oxa | | | | | |
| | zol-4-yl | | | | | |
| 35 | CHF₂CF₂ | Cl | CONH—CH₂ | 45 | 480.2 | 480.3 |
| 36 | 2-(2,5-dimethyl- | Cl | CONH—CH₂ | 29 | 475.3 | 475.2 |
| | oxa | | | | | |
| | zol-4-yl | | | | | |
| 37 | 2-furan-2-yl | Cl | CONH—CH₂ | 6 | 446.3 | 446.3 |
| 38 | 2-Fl-Ph | Cl | CONH—CH₂ | 31 | 474.3 | 474.2 |
| 39 | 2-MeO-Ph | Cl | CONH—CH₂ | 15 | 486.3 | 486.5 |
| 40 | 2-(1-methyl- | Cl | CONH—CH₂ | 13 | 459.3 | 459.3 |
| | 1H-pyrrol-2-yl) | | | | | |
| 41 | i-Bu | Cl | CH₂—CHF | 10 | 495.29 | 425.6 |
| 42 | i-Bu | Br | NHCO—CH₂ | 0.5 | 525.20 | 524.6 |
| | | | | | | (M − 1) |

Following the general procedure B2, three examples of esters were isolated

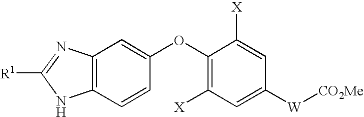

| Ex- | | | | | | |
|---|---|---|---|---|---|---|
| am- | | | | Yield | MW | M + 1 |
| ple | R¹ | X | W | (%) | (calc) | (found) |
| 43 | CHF₂CF₂ | Br | CONH—CH₂ | 3 | 583.1 | 584.0 |
| 44 | 2-(2,5-dimethyl- | Br | O—CH₂ | 12 | 551.2 | 552.3 |
| | oxa | | | | | |
| | zol-4-yl | | | | | |
| 45 | i-Bu | Br | NHCO—CH₂ | 1 | 539.23 | 540.6 |

### Example 46

### N-[3,5-Dibromo-4-(2-isobutyl-1H-benzoimidazol-5-yloxy)-phenyl]-acetamide

Following the general procedure B2 utilising intermediate 12 the main product isolated was N-[3,5-Dibromo-4-(2-isobutyl-1H-benzoimidazol-5-yloxy)-phenyl]-acetamide as a white solid. Yield: 23% M(+1)=482.0 (MW calc=481.19)

### Examples 47 and 48

### (R)-3-[3,5-Dibromo-4-(2-isobutyl-1H-benzoimidazol-5-yloxy)-phenyl]-2-fluoro-propionic acid

### (S)-3-[3,5-Dibromo-4-(2-isobutyl-1H-benzoimidazol-5-yloxy)-phenyl]-2-fluoro-propionic acid

Chiral HPLC Separation

Example 10, (3-{3,5-dibromo-4-[(2-isobutyl-1-isopropyl-1H-benzimidazol-6-yl)oxy]phenyl}-2-fluoropropanoic acid) is a racemic mixture. The single enantiomers were separated by chiral HPLC, providing examples 47 and 48.

HPLC Analyses

ReproSil Chiral-NR (4.6 mm id×250 mm, 8 μm (Dr. Maisch GmbH, Ammerbuch, Germany)) was used for HPLC analyses. Analyses were carried out using n-heptane: 2-propanol:trifluoroacetic acid (87:13:0.1%) as a mobile phase at a flow rate of 0.8 mL/min and room temperature. Detection was carried out at UV 282 nm. Under these conditions, the retention times were as follows:

Example 47: F1=84.7 min.

Example 48: F2=94.4 min

The peaks were tailing and not baseline separated

HPLC Preparative Separation

A column ReproSil Chiral-NR (20 mm id×250 mm, 8 μm (Dr. Maisch GmbH, Ammerbuch, Germany)) with a pre-column ReproSil Chiral-NR (30 mm id×20 mm, 8 μm (Dr. Maisch GmbH, Ammerbuch, Germany)) were used for HPLC preparative separation. The separations were carried out using n-heptane:2-propanol:trifluoroacetic acid (87:13:0.1%) as a mobile phase at a flow rate of 10.0 mL/min and room temperature. Detection was carried out at UV 282 nm. Under these conditions, the retention times were as follows:

Example 47: F1=118 min.

Example 48: F2=132 min

The peaks were tailing and not baseline separated

The pure fractions (determined by analytical HPLC analysis) were combined and evaporated. The EE of the combined fractions were determined as described above, giving a calculated EE of

EE Example 47=>99% (average of two injections)

EE Example 48=>98% (average of two injections)

The following compounds are further Examples of the invention:

| Example | Structure | Name |
|---|---|---|
| 49 | 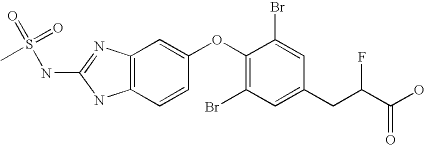 | 3-[3,5-Dibromo-4-(2- methanesulfonylamino- 1H-benzoimidazol-5- yloxy)-phenyl]-2- fluoro-propionic acid |
| 50 | 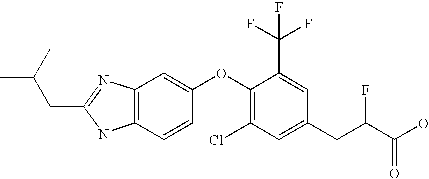 | 3-[3-Chloro-4-(2- isobutyl-1H- benzoimidazol-5- ylxoy)-5- trifluoromethyl- phenyl]-2-fluoro- propionic acid |
| 51 | 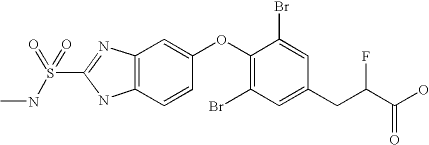 | 3-[3,5-Dibromo-4-(2- methylsulfamoyl-1H- benzoimidazol-5- yloxy)-phenyl]-2- fluoro-propionic acid |
| 52 | 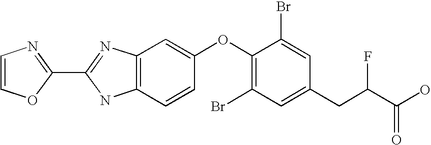 | 3-[3,5-Dibromo-4-(2- oxazol-2-yl-1H- benzoimidazol-5- yloxy)-phenyl]-2- fluoro-propionic acid |
| 53 | 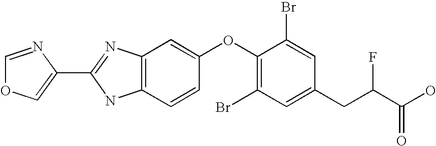 | 3-[3,5-Dibromo-4-(2- oxazol-4-yl-1H- benzoimidazol-5- yloxy)-phenyl]-2- fluoro-propionic acid |
| 54 | 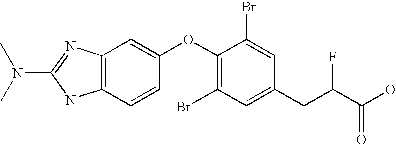 | 3-[3,5-Dibromo-4-(2- dimethylamino-1H- benzoimidazol-5- yloxy)-phenyl]-2- fluoro-propionic acid |
| 55 | 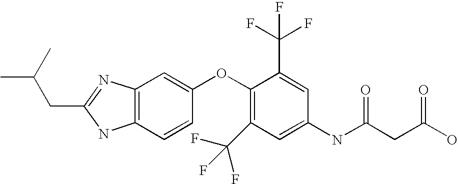 | N-[4-(2-Isobutyl-1H- benzoimidazol-5- yloxy)-3,5-bis- trifluoromethyl- phenyl]-malonamic acid |

TR Competition Binding Assay with Filter Separation

Compounds are tested for their ability to compete with the tracer ¹²⁵I-T3 for binding to full length hTRα and hTRβ. Receptor extracts and tracer are diluted in assay buffer (17 mM K₂HPO₄, 3 mM KH₂PO₄, 400 mM KCl, 1 mM MgCl₂, 0.5 mM EDTA and 8.7% glycerol). ¹²⁵I-T3 is diluted to a final concentration of 0.2 nM and receptor is diluted to reach a final count in Trilux Microbeta of approximately 10000 ccpm. Compounds are typically serially diluted in DMSO from DMSO stock solutions of 10 mM. To 96 well microtiter plates are 100 μl tracer, 4 μl test compound dilution series and 100 μl receptor dilution added. The assay plates are incubated at +4° C. overnight (app. 16 h incubation). Receptor bound and free tracer are separated over a glass fiber filter (FILTERMAT B, PerkinElmer) on a Tomtec Cellharvester with 18 mM K₂HPO₄, 2 mM KH2PO₄, 0.5 mM EDTA wash buffer. The filters are dried at 60° C. for 1 h and then merged with a scintillant wax (MELTILEX, PerkinElmer) on a Wallac Microsealer before measuring in a Trilux Microbeta. IC50s, the concentration test compound needed to decrease tracer binding by 50 percent, are generated via analysis of data in XLfit version 2.0 or later with a four parameter logistic model.

Compounds are considered to have activity as thyroid receptor-beta ligands if they have an IC₅₀ of 5000 nM or less. Preferred thyroid receptor-beta ligand compounds have an IC₅₀ of 500 nM or less, more preferably less than 100 nM, especially less than 30 nM. Particularly preferred as thyroid receptor-beta ligands are those compounds having an IC₅₀ of 10 nM or less.

Vector Constructs, Generation of Reporter Cell Lines (TRAF), and Assay Procedure.

The cDNAs encoding the full length human ThRα1 and ThRβ1 were cloned in the mammalian expression vector pMT-hGH. The pDR4-ALP reporter vector contains one copy of the direct repeat sequence AGGTCA nnnnAGGTCA, fused upstream of the core promoter sequences of the mouse mammary tumor virus long terminal repeat (MMTV), replacing the glucocorticoid response elements. The DR4-MMTV promoter fragment was then cloned in the 5′ end of the cDNA encoding human placental alkaline phosphatase (ALP), followed in the 3′-end by the polyA-signal sequence of the human growth hormone gene. Chinese hamster ovary (CHO) K1 cells (ATCC No. CCL 61) were transfected in two steps, first with the receptor expression vectors pMT-hThRα1 and pMT-THRβ1, respectively, and the drug resistance vector pSV2-Neo, and in the second step, with the reporter vector pDR4-ALP and the drug resistance vector pKSV-Hyg. Individual drug resistant clones were isolated and selected based on T3 inducibility. One stable reporter cell clone each of CHO/hThRα1 and CHO/hThRβ1 were chosen for further study in response to various thyroid hormone agonists.

Assay Procedure:

CHO/hThRα1 and CHO/hThRβ1 were seeded in growth medium (Coon's/F12, 10% L-3,5,3′-triiodothyronine and L-thyroxine depleted FCS, 2 mM L-glutamine) in 96-well plates at 20×10³ cells per well. After 24 hour incubation at 37° C. in humidified chambers, at 5% CO₂, conditioned medium was replaced by induction medium (OptiMEM, 2 mM L-glutamine, 50 μm/mL gentamycin) and cells were exposed to the test compounds at serial dilutions, at final DMSO concentration of 0.5% , or to serial dilution of T3 (positive control), to assess agonist activity of test compounds.

In order to examine antagonistic effect of test compounds, CHO/hThRα1 and CHO/hThRβ1 cells were exposed to serial dilution of the compounds in the presence of 1 nM T3 (CHO/hThRα1) or 3 nM T3 (CHO/hThRβ1).

After 48 hours incubation at 37° C. in humidified chambers at 5% CO₂ the level of alkaline phosphatase expressed and secreted into the cell culture medium was analyzed by chemiluminescence on MicroBeta Trilux.

Compounds are considered to be agonists of the thyroid receptor-beta ligands if they exhibit agonism of at least 20% and display no antagonism.

The assay procedure described above was carried out for the compounds of Examples 1 to 40, 43-44 and 47-48. Selected results for exemplary compounds are given in table below.

| | TR-β IC50 (nM) | β % agonism |
|---|---|---|
| Example 5 | 5.5 | 91 |
| Example 11 | 2.4 | 99 |
| Example 15 | 1.7 | 70 |
| Example 17 | 7.5 | 70 |
| Example 10 | 0.14 | 100 |
| Example 32 | 0.49 | 75 |

1. A method for the treatment or prophylaxis of a condition that may be treated with a thyroid receptor agonist or partial agonist in a mammal, which comprises administering to the mammal a therapeutically effective amount of a compound of formula (I) or a pharmaceutically acceptable ester, amide, solvate or salt thereof, including a salt of such an ester or amide, and a solvate of such an ester, amide or salt, 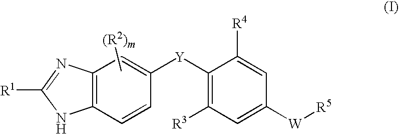
wherein:
R¹ is selected from halogen, N(R^{b})₂, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, —(CH₂)ₙ—CO—N(R^{a})₂, —CO₂H, C₁₋₈ alkyl, C₂₋₄ alkenyl, C₂₋₄ alkynyl, fluoromethyl, difluoromethyl, trifluoromethyl, C₃₋₆ cycloalkyl, C₃₋₆ cycloalkyl-C₁₋₃ alkyl, phenyl, benzyl and C₃₋₇ heterocyclyl, said alkyl, alkenyl or alkynyl groups or portions of groups optionally being substituted with 1, 2, 3, 4 or 5 groups each independently selected from halogen, hydroxy, C₁₋₄ alkylthio, N(R^{b})₂, phenyl, methoxy, halomethoxy, dihalomethoxy and trihalomethoxy; said cycloalkyl, phenyl, benzyl or heterocyclyl groups or portions of groups optionally being substituted with 1, 2 or 3 groups independently selected from halogen, hydroxy, C₁₋₄ alkyl, C₂₋₄ alkenyl, C₂₋₄ alkynyl, N(R^{b})₂, methoxy, haloC₁₋₄alkyl, dihaloC₁₋₄alkyl, trihaloC₁₋₄alkyl, halomethoxy, dihalomethoxy, and trihalomethoxy;
R^{a} is independently selected from C₁₋₄ alkyl, C₂₋₄ alkenyl, C₂₋₄ alkynyl, benzyl, heterocyclyl and phenyl, said phenyl group or portion of group optionally being substituted with 1, 2 or 3 groups independently selected from C₁₋₄ alkyl, halogen, hydroxy, methoxy, halomethoxy, dihalomethoxy, and trihalomethoxy; said alkyl, alkenyl, or alkynyl groups or portions of groups optionally being substituted with 1, 2 or 3 groups independently selected from halogen, hydroxy, methoxy, halomethoxy, dihalomethoxy, and trihalomethoxy;
n is 0, 1, 2 or 3;
Each R² is independently selected from halogen, hydroxy, cyano, C₁₋₄ alkoxy, C₁₋₄ alkyl and N(R^{b})₂, said alkyl or alkoxy groups or portions of groups optionally being substituted with 1, 2 or 3 groups selected from halogen, hydroxyl or C₁₋₄ alkoxy;
R^{b} is independently selected from hydrogen, C₁₋₄ alkyl, C₂₋₄ alkenyl, and C₂₋₄ alkynyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy;
m is 0, 1 or 2;
Y is selected from oxygen, methylene, sulphur, N(R^{b})₂, —S(O)— and —S(O)₂—;
R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;
W is selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NHC(O)—C₀₋₃ alkylene and C₁₋₃ alkylene C(O)NH—C₁₋₃ alkylene, said alkylene, alkenylene or alkynylene groups being straight chain, and said alkylene, alkenylene or alkynylene groups or portions of groups optionally being substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
or WR⁵ together form the group NHCOR^{d}
R^{c} is selected from hydrogen, C₁₋₄ alkyl, C₂₋₄ alkenyl, C₂₋₄ alkynyl, fluoromethyl, difluoromethyl and trifluoromethyl;
R⁵ is selected from —CO₂R^{d}, —CONHR^{d}, —PO(OR^{d})₂, —PO(OR^{d})NH₂, —SO₂OR^{d}, —COCO₂R^{d}, —CONR^{d}OR^{d}, —SO₂NHR^{d}, —NHSO₂R^{d}, —CONHSO₂R^{d}, and —SO₂NHCOR^{d};
Each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen and C₁₋₄ alkyl;
with the proviso that when Y is oxygen, W is methylene, m is 0, R³ and R⁴ are both chlorine, and R⁵ is CO₂H, R¹ is not isopropyl;
and with the further proviso that when Y is oxygen, W is methylene, m is 0, R³ and R⁴ are both bromine, and R⁵ is CO₂H, R¹ is not methyl.

2. A compound of formula (Ia) or a pharmaceutically acceptable ester, amide, solvate or salt thereof, including a salt of such an ester or amide, and a solvate of such an ester, amide or salt, 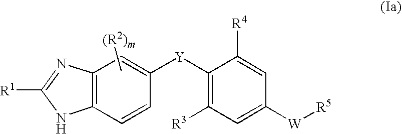
wherein:
R¹ is selected from halogen, N(R^{b})₂, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, —(CH₂)ₙ—CO—N(R^{a})₂, —CO₂H, C₁₋₈ alkyl, C₂₋₄ alkenyl, C₂₋₄ alkynyl, fluoromethyl, difluoromethyl, trifluoromethyl, C₃₋₆ cycloalkyl, C₃₋₆ cycloalkyl-C₁₋₃ alkyl, phenyl, benzyl and C₃₋₇ heterocyclyl, said alkyl, alkenyl or alkynyl groups or portions of groups optionally being substituted with 1, 2, 3, 4 or 5 groups each independently selected from halogen, hydroxy, C₁₋₄ alkylthio, N(R^{b})₂, phenyl, methoxy, halomethoxy, dihalomethoxy and trihalomethoxy; said cycloalkyl, phenyl, benzyl or heterocyclyl groups or portions of groups optionally being substituted with 1, 2 or 3 groups independently selected from halogen, hydroxy, C₂₋₄ alkyl, C₂₋₄ alkenyl, C₂₋₄ alkynyl, N(R^{b})₂, methoxy, haloC₁₋₄alkyl, dihaloC₁₋₄alkyl, trihaloC₁₋₄alkyl, halomethoxy, dihalomethoxy, and trihalomethoxy;
R^{a} is independently selected from C₁₋₄ alkyl, C₂₋₄ alkenyl, C₂₋₄ alkynyl, benzyl, heterocyclyl and phenyl, said phenyl group or portion of group optionally being substituted with 1, 2 or 3 groups independently selected from C₁₋₄ alkyl, halogen, hydroxy, methoxy, halomethoxy, dihalomethoxy, and trihalomethoxy; said alkyl, alkenyl, or alkynyl groups or portions of groups optionally being substituted with 1, 2 or 3 groups independently selected from halogen, hydroxy, methoxy, halomethoxy, dihalomethoxy, and trihalomethoxy;
n is 0, 1, 2 or 3;
Each R² is independently selected from halogen, hydroxy, cyano, C₁₋₄ alkoxy, C₁₋₄ alkyl and N(R^{b})₂, said alkyl or alkoxy groups or portions of groups optionally being substituted with 1, 2 or 3 groups selected from halogen, hydroxyl or C₁₋₄ alkoxy;
R^{b} is independently selected from hydrogen, C₁₋₄ alkyl, C₂₋₄ alkenyl, and C₂₋₄ alkynyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy;
m is 0, 1 or 2;
Y is selected from oxygen, methylene, N(R^{b})₂, sulphur, —S(O)— and —S(O)₂—;
R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;
W is selected from
C₁ alkylene substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
Straight chain C₂₋₃ alkylene substituted with 1 or 2 groups selected from mercapto, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
C₂₋₃ alkenylene, C₂₋₃ alkynylene, N(R^{c})-C₁₋₃ alkylene, C(O)-C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NHC(O)—C₀₋₃ alkylene and C₁₋₃ alkyleneC(O)NH—C₁₋₃ alkylene, said alkylene, alkenylene or alkynylene groups being straight chain, and said alkylene, alkenylene or alkynylene groups or portions of groups optionally being substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl,
trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
R^{c} is selected from hydrogen, C₁₋₄ alkyl, C₂₋₄ alkenyl, C₂₋₄ alkynyl, fluoromethyl, difluoromethyl and trifluoromethyl;
R⁵ is selected from —CO₂R^{d}, —CONHR^{d}, —PO(OR^{d})₂, —PO(OR^{d})NH₂, —SO₂OR^{d}, —COCO₂R^{d}, —CONR^{d}OR^{d}, —SO₂NHR^{d}, —NHSO₂R^{d}, —CONHSO₂R^{d}, and —SO₂NHCOR^{d};
or WR⁵ together form the group NHCOR^{d}
Each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen and C₁₋₄ alkyl.

3. A compound of formula (Ib) or a pharmaceutically acceptable ester, amide, solvate or salt thereof, including a salt of such an ester or amide, and a solvate of such an ester, amide or salt, 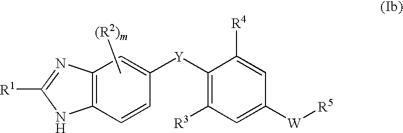
wherein:
R¹ is selected from
C₁₋₄ alkyl substituted with one group independently selected from halogen, hydroxy, C₁₋₄ alkylthio, N(R^{b})₂, methoxy, halomethoxy, dihalomethoxy and trihalomethoxy, and optionally substituted with 1, 2, 3 or 4 additional groups each independently selected from halogen, hydroxy, C₁₋₄ alkylthio, N(R^{b})₂, phenyl, methoxy, halomethoxy, dihalomethoxy and trihalomethoxy;
phenyl or C₅₋₇ heteroaryl, said phenyl or C₅₋₇ heteroaryl group being substituted with one group independently selected from chlorine, bromine, iodine, C₁₋₄ alkyl, C₂₋₄ alkenyl, C₂₋₄ alkynyl, haloC₁₋₄alkyl, dihaloC₁₋₄alkyl, trihaloC₁₋₄alkyl, halomethoxy, dihalomethoxy, and trihalomethoxy, and optionally substituted with 1 or 2 additional groups each independently selected from halogen, hydroxy, C₁₋₄ alkyl, C₂₋₄ alkenyl, C₂₋₄ alkynyl, N(R^{b})₂, methoxy, haloC₁₋₄alkyl, dihaloC₁₋₄alkyl, trihaloC₁₋₄alkyl, halomethoxy, dihalomethoxy, and trihalomethoxy;
halogen, N(R^{b})₂, —(CH₂)ₙ—NH-SO₂—R^{a}, —(CH₂)ₙ—SO₂—NH-R^{a}, —(CH₂)ₙ—NH—CO—R^{a}, —(CH₂)ₙ—CO—NH—R^{a}, C₅₋₈ alkyl, C₂₋₄ alkenyl, C₂₋₄ alkynyl, fluoromethyl, difluoromethyl, trifluoromethyl, C₃₋₆ cycloalkyl, C₃₋₆ cycloalkyl-C₁₋₃ alkyl, benzyl and C₃₋₄ heterocyclyl, C₅₋₇ heterocycloalkyl, said alkyl, alkenyl or alkynyl groups or portions of groups optionally being substituted with 1, 2, 3, 4 or 5 groups each independently selected from halogen, hydroxy, C₁₋₄ alkylthio, N(R^{b})₂, phenyl, methoxy, halomethoxy, dihalomethoxy and trihalomethoxy; said cycloalkyl, benzyl, heterocyclyl or heterocycloalkyl groups or portions of groups optionally being substituted with 1, 2 or 3 groups independently selected from halogen, hydroxy, C₁₋₄ alkyl, C₂₋₄ alkenyl, C₂₋₄ alkynyl, N(R^{b})₂, methoxy, halomethoxy, dihalomethoxy, and trihalomethoxy;
Ra is independently selected from C₁₋₄ alkyl, C₂₋₄ alkenyl, C₂₋₄ alkynyl, benzyl, heterocyclyl and phenyl, phenyl group or portion of group optionally being substituted with 1, 2 or 3 groups independently selected from C₁₋₄ alkyl, halogen, hydroxy, methoxy, halomethoxy, dihalomethoxy, and trihalomethoxy; said alkyl, alkenyl, or alkynyl groups or portions of groups optionally being substituted with 1, 2 or 3 groups independently selected from halogen, hydroxy, methoxy, halomethoxy, dihalomethoxy, and trihalomethoxy;
n is 0, 1, 2 or 3;
Each R² is independently selected from halogen, hydroxy, cyano, C₁₋₄ alkoxy, C₁₋₄ alkyl and N(R^{b})₂, said alkyl or alkoxy groups or portions of groups optionally being substituted with 1, 2 or 3 groups selected from halogen, hydroxyl or C₁₋₄ alkoxy;
R^{b} is independently selected from hydrogen, C₁₋₄ alkyl, C₂₋₄ alkenyl, and C₂₋₄ alkynyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy;
m is 0, 1 or 2;
Y is selected from oxygen, methylene, N(R^{b})₂, sulphur, —S(O)— and —S(O)₂—;
R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;
W is selected from C₁₋₃ alkylene, and C₂₋₃ alkylene substituted with 1 or 2 groups selected from hydroxy and amino;
R⁵ is selected from —CO₂R^{d}, —CONHR^{d}, —PO(OR^{d})₂, -PO(ORd)NH₂, —SO₂OR^{d}, —COCO₂R^{d}, —CONR^{d}OR^{d}, —SO₂NHR^{d}, —NHSO₂R^{d}, —CONHSO₂R^{d}, and —SO₂NHCOR^{d};
or WR⁵ together form the group NHCOR^{d}
Each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen and C₁₋₄ alkyl.

4. A compound of formula (Ic) or a pharmaceutically acceptable ester, amide, solvate or salt thereof, including a salt of such an ester or amide, and a solvate of such an ester, amide or salt, 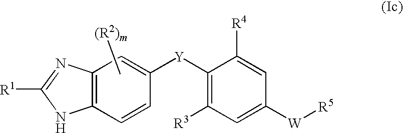
wherein:
R¹ is selected from C₁₋₄ alkyl, phenyl, and C₅₋₇ heteroaryl, said alkyl groups optionally being substituted with 1, 2 or 3 phenyl groups; said phenyl or heteroaryl groups optionally being substituted with 1, 2 or 3 groups independently selected from fluorine, hydroxy, methoxy and N(R^{b})₂;
Each R² is independently selected from halogen, cyano, hydroxy, C₁₋₄ alkoxy, C₁₋₄ alkyl and N(R^{b})₂, said alkyl or alkoxy groups or portions of groups optionally being substituted with 1, 2 or 3 groups selected from halogen, hydroxyl or C₁₋₄ alkoxy;
R^{b} is independently selected from hydrogen, C₁₋₄ alkyl, C₂₋₄ alkenyl, and C₂₋₄ alkynyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy;
m is 0, 1 or 2;
Y is selected from oxygen, methylene, N(R^{b})₂, sulphur, —S(O)— and —S(O)₂—;
R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;
W is selected from C₁₋₃ alkylene, and C₂₋₃ alkylene substituted with 1 or 2 groups selected from hydroxy and amino;
R⁵ is selected from —CO₂R^{d}, —CONHR^{d}, —PO(OR^{d})₂, —PO(OR^{d})NH₂, —SO₂OR^{d}, —COCO₂R^{d}, —CONR₂OR^{d}, —SO₂NHR^{d}, —NHSO₂R^{d}, —CONHSO₂R^{d}, and —SO₂NHCOR^{d};
Each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen and C₁₋₄ alkyl;
with the proviso that when Y is oxygen, W is methylene, m is 0, R³ and R⁴ are both chlorine, and R⁵ is CO₂H, R¹ is not isopropyl;
and with the further proviso that when Y is oxygen, W is methylene, m is 0, R³ and R⁴ are both bromine, and R⁵ is CO₂H, R¹ is not methyl.

5. (canceled)

6. (canceled)

7. (canceled)

8. (canceled)

9. A pharmaceutical composition comprising a compound of formula (Ia) as defined in claim 2 or a pharmaceutically acceptable ester, amide, solvate or salt thereof, including a salt of such an ester or amide, and including a solvate of such an ester, amide or salt, and a pharmaceutically acceptable excipient.

10. A pharmaceutical composition as claimed in claim 9 further comprising an additional therapeutic agent selected from cholesterol/lipid lowering agents, hypolipidemic agents, anti-atherosclerotic agents, anti-diabetic agents, anti-osteoporosis agents, anti-obesity agents, growth promoting agents, anti-inflammatory agents, anti-anxiety agents, anti-depressants, anti-hypertensive agents, cardiac glycosides, appetite suppressants, bone resorption inhibitors, thyroid mimetics, anabolic agents, anti-tumor agents and retinoids.

11. Use of a compound of formula (I) as defined in claim 1 in labelled form as a diagnostic agent for the diagnosis of conditions that may be treated with a thyroid receptor agonist or partial agonist.

12. Use of a compound of formula (Ia) as defined in claim 2 or a labelled form of such a compound as a reference compound in a method of identifying ligands for the thyroid hormone receptor.

13. A a method as claimed in claim 1, wherein the condition that may be treated with a thyroid receptor agonist or partial agonist is selected from (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes; (7) depression; (8) osteoporosis (especially in combination with a bone resorption inhibitor); (9) goiter; (10) thyroid cancer; and (11) glaucoma.

14. A method for preparing a compound of formula (Ia), as defined in claim 2 wherein Y is oxygen, sulphur or N(R^{b}) comprising a step of reacting
a compound of formula (II) 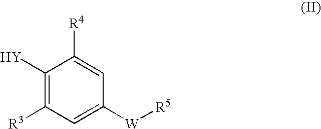
wherein W, R³, R⁴, and R⁵ are as defined in claim 2 and Y is oxygen, sulphur or N(R^{b})
with a compound of formula (III) 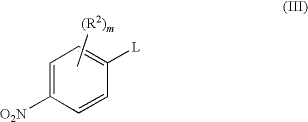
wherein R² and m are as defined in claim 2 and L is a suitable leaving group, optionally in the presence of a suitable base and, optionally, in the presence of copper powder, followed optionally by interconversion to another compound of formula (Ia) as defined in claim 2.

15. A method for preparing a compound of formula (Ia) as defined in claim 2 wherein Y is oxygen, sulphur, methylene or N(R^{b}) comprising a step of reacting
a compound of formula (IV) 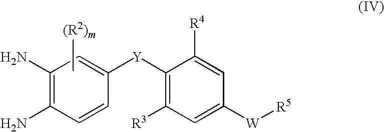
wherein R², R³, R⁴, R⁵, m and W are as defined in claim 2 and Y is oxygen, sulphur, methylene or N(R^{b})
with a compound of formula (V) 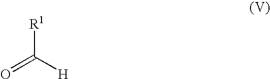
wherein R¹ is as defined in claim 2 in the presence of a suitable acid and, followed optionally by interconversion to another compound of formula (Ia) as defined in claim 2 wherein Y is oxygen.

16. A method for preparing a compound of formula (Ia) as defined in claim 2 wherein Y is oxygen, sulphur, methylene or N(R^{b}) comprising a step of reacting
a compound of formula (VI) 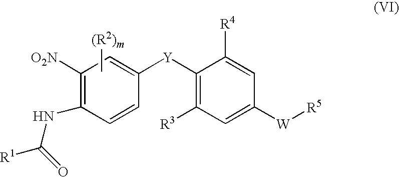
wherein R¹, R², R³, R⁴, R⁵, m and W are as defined in claim 2 and Y is oxygen, sulphur, methylene or N(R^{b})
with a suitable reducing agent followed by heating in the presence of a suitable acid and, followed optionally by interconversion to another compound of formula (Ia) as defined in claim 2 wherein Y is oxygen, sulphur, methylene or N(R^{b}).

17. A method for preparing a compound of formula (Ia) as defined in claim 2 wherein Y is oxygen, sulphur, methylene or N(R^{b}) comprising a step of reacting
a compound of formula (X) 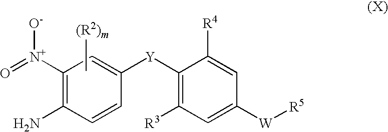
wherein R², R³, R⁴, R⁵, m and W are as defined in claim 2 and Y is oxygen, sulphur, methylene or N(R^{b})
with a compound of formula (XI) 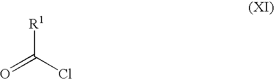
wherein R¹ is as defined in claim 2 in the presence of a suitable reducing agent and followed by heating in the presence of a suitable acid and, followed optionally by interconversion to another compound of formula (Ia) as defined in claim 2 wherein Y is oxygen, sulphur, methylene or N(R^{b}).

18. A use as claimed in claim 11, wherein the condition that may be treated with a thyroid receptor agonist or partial agonist is selected from (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes; (7) depression; (8) osteoporosis (especially in combination with a bone resorption inhibitor); (9) goiter; (10) thyroid cancer; and (11) glaucoma.

19. A pharmaceutical composition comprising a compound of formula (Ib) as defined in claim 3 or a pharmaceutically acceptable ester, amide, solvate or salt thereof, including a salt of such an ester or amide, and including a solvate of such an ester, amide or salt, and a pharmaceutically acceptable excipient.

20. A pharmaceutical composition as claimed in claim 19 further comprising an additional therapeutic agent selected from cholesterol/lipid lowering agents, hypolipidemic agents, anti-atherosclerotic agents, anti-diabetic agents, anti-osteoporosis agents, anti-obesity agents, growth promoting agents, anti-inflammatory agents, anti-anxiety agents, anti-depressants, anti-hypertensive agents, cardiac glycosides, appetite suppressants, bone resorption inhibitors, thyroid mimetics, anabolic agents, anti-tumor agents and retinoids.

21. Use of a compound of formula (Ib) as defined in claim 3 or a labelled form of such a compound as a reference compound in a method of identifying ligands for the thyroid hormone receptor.

22. A method for preparing a compound of formula (Ib) as defined in claim 3 wherein Y is oxygen, sulphur or N(R^{b}) comprising a step of reacting
a compound of formula (II) 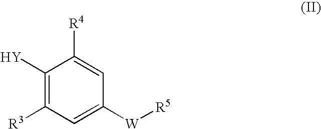
wherein W, R³, R⁴, and R⁵ are as defined in claim 3 and Y is oxygen, sulphur or N(R^{b})
with a compound of formula (III) 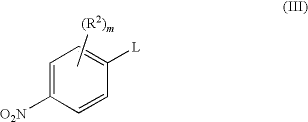
wherein R² and m are as defined in claim 3 and L is a suitable leaving group, optionally in the presence of a suitable base and, optionally, in the presence of copper powder, followed optionally by interconversion to another compound of formula (Ib) as defined in claim 3.

23. A method for preparing a compound of formula (Ib) as defined in claim 3wherein Y is oxygen, sulphur, methylene or N(R^{b}) comprising a step of reacting
a compound of formula (IV) 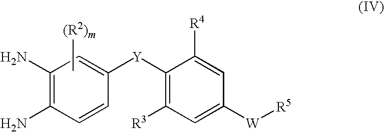
wherein R², R³, R⁴, R⁵, m and W are as defined in claim 3 and Y is oxygen, sulphur, methylene or N(R^{b})
with a compound of formula (V) 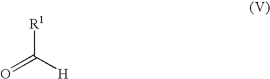
wherein R¹ is as defined in claim 3 in the presence of a suitable acid and, followed optionally by interconversion to another compound of formula (Ib) as defined in claim 3 wherein Y is oxygen.

24. A method for preparing a compound of formula (Ib) as defined in claim 3 wherein Y is oxygen, sulphur, methylene or N(R^{b}) comprising a step of reacting
a compound of formula (VI) 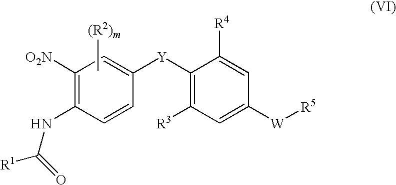
wherein R¹, R², R³, R⁴, R⁵, m and W are as defined in claim 3 and Y is oxygen, sulphur, methylene or N(R^{b})
with a suitable reducing agent followed by heating in the presence of a suitable acid and, followed optionally by interconversion to another compound of formula (Ib) as defined in claim 3 wherein Y is oxygen, sulphur, methylene or N(R^{b}).

25. A method for preparing a compound of formula (Ib) as defined in claim 3 wherein Y is oxygen, sulphur, methylene or N(R^{b}) comprising a step of reacting
a compound of formula (X) 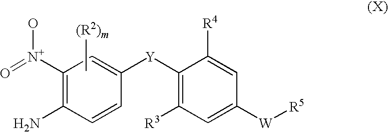
wherein R², R³, R⁴, R⁵, m and W are as defined in claim 3 and Y is oxygen, sulphur, methylene or N(R^{b})
with a compound of formula (XI) 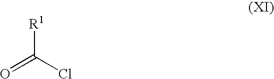
wherein R¹ is as defined in claim 3
in the presence of a suitable reducing agent and followed by heating in the presence of a suitable acid and, followed optionally by interconversion to another compound of formula (Ib) as defined in claim 3 wherein Y is oxygen, sulphur, methylene or N(R^{b}).


SEQUENCE LISTING (1 sequence)

SEQ ID NO: 1 - DNA, 16 bp, Artificial Sequence
  Direct repeat sequence in pDR4-ALP reporter vector
aggtcannnnaggtca